(19) United States
(12) Patent Application Publication
(10) Pub. No.: US 2017/0029698 A1
(43) Pub. Date: Feb. 2, 2017

(54) ANTIOXIDANT FOR LIQUID CRYSTAL COMPOSITION, LIQUID CRYSTAL COMPOSITION, AND LIQUID CRYSTAL DISPLAY DEVICE USING LIQUID CRYSTAL COMPOSITION

(71) Applicant: DIC Corporation, Tokyo (JP)

(72) Inventors: Go Sudo, Kita-adachi-gun (JP); Yasuo Umezu, Kita-adachi-gun (JP)

(73) Assignee: DIC Corporation, Tokyo (JP)

(21) Appl. No.: 15/106,448

(22) PCT Filed: Dec. 17, 2014
(86) PCT No.: PCT/JP2014/083446

§ 371 Date: Jun. 20, 2016

(30) Foreign Application Priority Data
Dec. 25, 2013 (JP) 2013-266998

(51) Int. Cl. C09K 15/08; G02F 1/1368; C07C 39/16; G02F 1/1343; C09K 19/32; C09K 19/54

(52) U.S. Cl. CPC C09K 15/08; C09K 19/322; C09K 19/54; C09K 19/542; C07C 39/16; G02F 1/13439; G02F 1/1368; C09K 2019/548; C09K 2019/0466

(57) ABSTRACT

The present invention is to provide an antioxidant for liquid crystal composition which contains one or more kinds of liquid crystal compounds having a positive dielectric anisotropy of 4 or more as an absolute value at 3% or more and is represented by Formula (II-1)

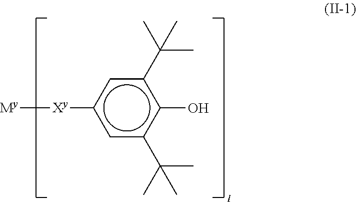

(in the Formula (II-1), M^{y} represents a hydrocarbon having from 1 to 25 carbon atoms, a 1,4-phenylene group, or a trans-1,4-cyclohexylene group, X^{y} represent an alkylene group having from 1 to 15 carbon atoms, —OCH₂—, —CH₂O—, —COO—, —OCO—, —CF₂O—, —OCF₂—, —CF₂CF₂—, —CH═CH—COO—, —CH═CH—OCO—, —COO—CH═CH—, —OCO—CH═CH—, —CH═CH—, —C≡C—, a singlebond, a 1,4-phenylene group, or a trans-1,4-cyclohexylene group, and l represents an integer from 2 to 6), a liquid crystal composition containing the antioxidant, and a liquid crystal display device using the liquid crystal composition.

### TECHNICAL FIELD

The present invention relates to an antioxidant for liquid crystal composition useful as a liquid crystal display material, a nematic liquid crystal composition having a positive dielectric anisotropic (Δ∈) value, and a liquid crystal display device using the nematic liquid crystal composition.

### BACKGROUND ART

Liquid crystal display devices are used in a timepiece, an electronic calculator, various kinds of measuring instruments, a panel for motor vehicle, a word processor, an electronic notebook, a printer, a computer, a television, a timepiece, and an advertising display board. As the liquid crystal display mode, there is typically a TN (twisted nematic) type, a STN (super twisted nematic) type, a vertical alignment type using a TFT (thin film transistor), or a horizontal alignment type such as an IPS (in-plane switching) type or a FFS (fringe field switching) type. The liquid crystal compositions used in these liquid crystal display devices are required to be stable with respect to external stimuli such as moisture, air, heat, and light, also show a liquid crystal phase in a wide temperature range including room temperature as possible, to exhibit low viscosity, and to have a low driving voltage. Furthermore, the liquid crystal compositions are composed of from several to several tens kinds of compounds in order to obtain an optimal dielectric anisotropic (Δ∈) or/and refractive index anisotropic (Δn) value as individual display devices.

A liquid crystal composition having a negative Δ∈ value is used in the vertical alignment (VA) type display and a liquid crystal composition having a positive Δ∈ value is used in the horizontal alignment type display such as a TN type, a STN type, or an IPS (in-plane switching) type. In addition, a driving mode has also been reported in which a liquid crystal composition having a positive Δ∈ value is vertically aligned when the voltage is not applied and the display is performed by applying a horizontal electric field, and thus a liquid crystal composition having a positive Δ∈ value has been increasingly required. Meanwhile, low voltage driving, high speed response, and a wide operating temperature range are required in all driving modes. In other words, a positive Δ∈ value, a great absolute Δ∈ value, a low viscosity (η), and a high nematic phase-isotropic liquid phase transition temperature (Tni) are required. In addition, it is required to adjust Δn of the liquid crystal composition into a suitable range in accordance with the cell gap in setting Δn×d of the product of Δn and the cell gap (d). In addition, a liquid crystal composition having a low rotational viscosity (γ1) is required since high speed response is important in the case of applying the liquid crystal display device to a television, or the like.

As the configuration of a liquid crystal composition intended to exhibit high speed response, for example, a liquid crystal composition in which a compound represented by Formula (A-1) or (A-2) that is a liquid crystal compound having a positive Δ∈ value and (B) of a liquid crystal compound having a neutral Δ∈ value are used in combination is disclosed. The features of these liquid crystal compositions that the liquid crystal compound having a positive Δ∈ value has a —CF₂O— structure or the liquid crystal compound having a neutral Δ∈ value has an alkenyl group are widely known in this liquid crystal composition field. (Patent Literatures 1 to 4)

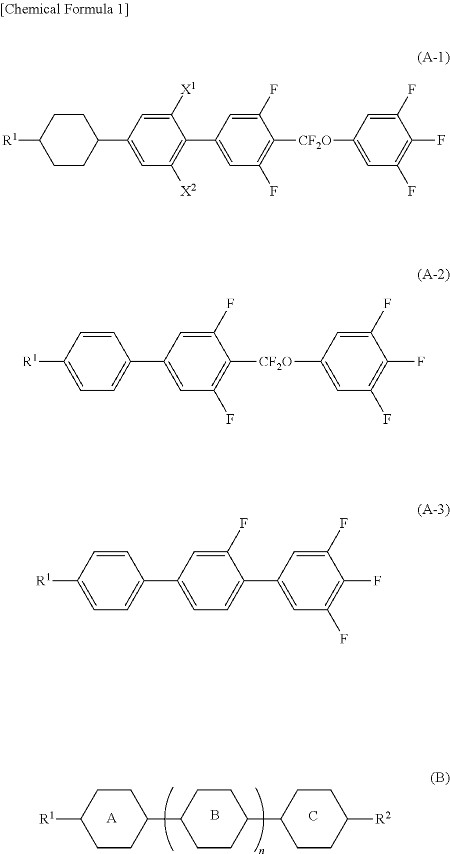

In addition, for example, as disclosed in Patent Literature 5, an antioxidant is generally mixed in the liquid crystal composition for the purpose of preventing a decrease in specific resistance due to heating in the air or maintaining a great voltage holding ratio in the vicinity of the upper limit temperature of the nematic phase after a long term operation of the liquid crystal display device.

### CITATION LIST

### Patent Literature

Patent Literature 1: JP 2008-037918 A

Patent Literature 2: JP 2008-038018 A

Patent Literature 3: JP 2010-275390 A

Patent Literature 4: JP 2011-052120 A

Patent Literature 5: JP 2013-173915 A

### SUMMARY OF INVENTION

### Technical Problem

The liquid crystal compositions of Patent Literatures 1 to 5 are regarded to be equipped with at least one characteristic among the characteristics such as a high upper limit temperature of the nematic phase, a low lower limit temperature of the nematic phase, a low viscosity, a great optical anisotropy, a great dielectric anisotropy, a great specific resistance, high stability with respect to ultraviolet light, and high stability with respect to heat.

However, the extension of applications of the liquid crystal display devices in recent years has also led a great change in usage or manufacturing methods thereof, and it is required to optimize the characteristics other than the above basic characteristics as known in the prior art. In other words, a VA-type or IPS-type liquid crystal display device is now widely used, and even an ultra-large size display device of 50-inch or more is now commercialized with regard to the size. The mainstream of injection method of the liquid crystal composition into a substrate is also changed from a vacuum injection method of the prior art to a one drop fill (ODF) method in association with such an increase in substrate size of the liquid crystal display device, but a problem that the drop mark when the liquid crystal composition is dropped on the substrate causes a decrease in display quality has been revealed. Furthermore, in the manufacturing process of a liquid crystal display device by the ODF method, it is required to drop an optimal injection amount of liquid crystal according to the size of the liquid crystal display device. Hence, the balance between the refractive index and the driving electric field of the liquid crystal display device that has been designed in advance is disturbed when the injection amount of liquid crystal is greatly deviated from the optimal value, and thus display defects such as spot occurrence or contrast defects occur. In particular, it is difficult to control the deviation from the optimal value within a certain range in a small-sized liquid crystal display device that is frequently used in smartphones which are recently prevalent since the optimal injection amount of liquid crystal is small. Consequently, the liquid crystal composition is also required to have performance, for example, capable of being hardly affected by a rapid pressure change in the dropping apparatus occurring at the time of liquid crystal dropping or an impact and continuously and stably dropping the liquid crystal for a long period of time in order to hold a high yield of the liquid crystal display device.

As described above, it has been required to develop a liquid crystal composition used in an active matrix driving liquid crystal display device that is driven by a TFT device or the like in consideration of the manufacturing method of the liquid crystal display device in addition to a high specific resistance or a high voltage holding ratio that has been important in the prior art or the characteristic of being stable with respect to the external stimulus such as light or heat while maintaining the characteristic or performance desired as a liquid crystal display device such as high speed response performance. Hence, the antioxidant to be added to such a liquid crystal composition is required to maintain the characteristic of the liquid crystal composition.

However, it has been confirmed that a new problem occurs that ionic impurities derived from the antioxidant that is present in the liquid crystal layer of a liquid crystal display device not only become a factor to lower the voltage holding ratio but the ionic impurities also move in a plane along the electric field formed in the liquid crystal layer so that the ionic impurities derived from the antioxidant accumulate at a particular part (hot spot) such as the vicinity of the electrode and this region having the ionic impurities accumulated is visible from the outside as a (linear) residual image.

Accordingly, the present invention is to solve the above problem and also to provide an antioxidant that is used in a liquid crystal composition having a positive dielectric anisotropy (Δ∈) and maintains the characteristics of a liquid crystal composition which has a liquid crystal phase in a wide temperature range, exhibits low viscosity and favorable solubility at a low temperature, has a high specific resistance or a high voltage holding ratio, or is stable with respect to heat or light, and a liquid crystal composition containing the antioxidant. Furthermore, the present invention is to provide a liquid crystal display device of a TN type, an IPS type, a FFS type, or the like which exhibits excellent display quality and hardly causes display defects such as burn-in or a drop mark by use of this in a favorable yield.

### Solution to Problem

The present inventors have investigated various kinds of liquid crystal compounds and various kinds of chemical substances and found out that the above problem can be solved, thereby completing the present invention.

There are provided an antioxidant for liquid crystal composition including one or more kinds of liquid crystal compounds having a positive dielectric anisotropy of 4 or more as an absolute value at 3% or more, wherein the antioxidant for liquid crystal composition is used in a liquid crystal composition, and the antioxidant for liquid crystal composition is represented by Formula (II-1):

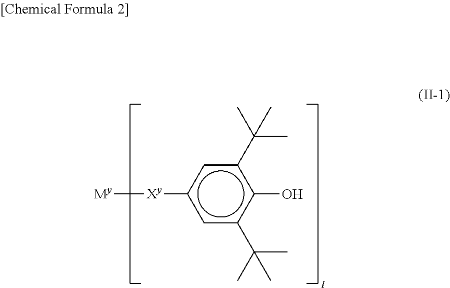

(in Formula (II-1), M^{y} represents a hydrocarbon having from 1 to 25 carbon atoms (one or two or more —CH₂— in the hydrocarbon may be substituted with —O—, —CO—, —COO—, or —OCO— so as not to be directly adjacent to an oxygen atom), a 1,4-phenylene group, or a trans-1,4-cyclohexylene group, wherein an arbitrary hydrogen atom in the 1,4-phenylene group may be substituted with a fluorine atom, X^{y}'s may be the same as or different from one another and represent an alkylene group having from 1 to 15 carbon atoms (one or two or more —CH₂— in the alkylene group may be substituted with —O—, —CO—, —COO—, or —OCO— so as not to be directly adjacent to an oxygen atom), —OCH₂—, —CH₂O—, —COO—, —OCO—, —CF₂O—, —OCF₂—, —CF₂CF₂—, —CH═CH—COO—, —CH═CH—OCO—, —COO—CH═CH—, —OCO—CH═CH—, —CH═CH—, —C≡C—, a single bond, a 1,4-phenylene group, or a trans-1,4-cyclohexylene group, wherein an arbitrary hydrogen atom in the 1,4-phenylene group may be substituted with a fluorine atom, and l represents an integer from 2 to 6), a liquid crystal composition containing the antioxidant, and a liquid crystal display device using the liquid crystal composition.

### Advantageous Effects of Invention

The antioxidant for liquid crystal composition according to the present invention can maintain the solubility of the entire liquid crystal composition at a low temperature even when being mixed with a liquid crystal compound.

The liquid crystal composition according to the present invention can obtain a significantly low viscosity or rotational viscosity, exhibits favorable solubility at a low temperature, has a high specific resistance or a high voltage holding ratio, and has a significantly small change caused by heat or light, and thus practicality of the product is high, and the liquid crystal display device of a TN type, an IPS type, or the like using this can achieve high speed response, has suppressed display defects such as a drop mark or a linear residual image, and is thus significantly useful.

### BRIEF DESCRIPTION OF DRAWINGS

FIG. 1 is a view schematically illustrating the configuration of a liquid crystal display device according to the present invention.

FIG. 2 is an enlarged plane view of the region II in the electrode layer that is formed on the substrate in FIG. 1 and includes a thin film transistor.

FIG. 3 is a cross-sectional view of the liquid crystal display device that is illustrated in FIG. 1 and cut in the line III-III direction in FIG. 2.

### DESCRIPTION OF EMBODIMENTS

According to the present invention, there is provided an antioxidant for liquid crystal composition including one or more kinds of liquid crystal compounds having a positive dielectric anisotropy of 4 or more as an absolute value at 3% or more, wherein the antioxidant for liquid crystal composition is used in a liquid crystal composition, and the antioxidant for liquid crystal composition is represented by Formula (II-1):

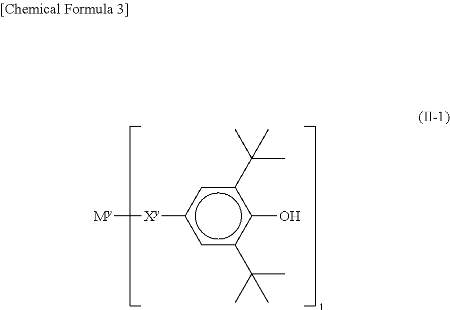

(in Formula (II-1), M^{y} represents a hydrocarbon having from 1 to 25 carbon atoms (one or two or more —CH₂— in the hydrocarbon may be substituted with —O—, —CO—, —COO—, or —OCO— so as not to be directly adjacent to an oxygen atom), a 1,4-phenylene group, or a trans-1,4-cyclohexylene group, wherein an arbitrary hydrogen atom in the 1,4-phenylene group may be substituted with a fluorine atom, X^{y}'s may be the same as or different from one another and represent an alkylene group having from 1 to 15 carbon atoms (one or two or more —CH₂— in the alkylene group may be substituted with —O—, —CO—, —COO—, or —OCO— so as not to be directly adjacent to an oxygen atom), —OCH₂—, —CH₂O—, —COO—, —OCO—, —CF₂O—, —OCF₂—, —CF₂CF₂—, —CH═CH—COO—, —CH═CH—OCO—, —COO—CH═CH—, —OCO—CH═CH—, —CH═CH—, —C≡C—, a single bond, a 1,4-phenylene group, or a trans-1,4-cyclohexylene group, wherein an arbitrary hydrogen atom in the 1,4-phenylene group may be substituted with a fluorine atom, and l represents an integer from 2 to 6).

The antioxidant according to the present invention can maintain the solubility of the entire liquid crystal composition at a low temperature even when being mixed with a liquid crystal compound.

The antioxidant in the present invention contains one kind or two or more kinds of compounds represented by Formula (II-1):

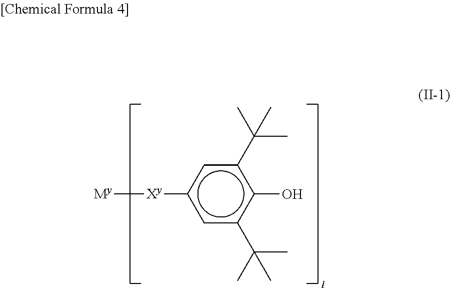

(in Formula (II-1) above, M^{y} represents a hydrocarbon having from 1 to 25 carbon atoms (one or two or more —CH₂— in the hydrocarbon may be substituted with —O—, —CO—, —COO—, or —OCO— so as not to be directly adjacent to an oxygen atom), a 1,4-phenylene group, or a trans-1,4-cyclohexylene group, in which an arbitrary hydrogen atom in the 1,4-phenylene group may be substituted with a fluorine atom, X^{y}'s may be the same as or different from one another and represent an alkylene group having from 1 to 15 carbon atoms (one or two or more —CH₂— in the alkylene group may be substituted with —O—, —CO—, —COO—, or —OCO— so as not to be directly adjacent to an oxygen atom), —OCH₂—, —CH₂O—, —COO—, —OCO—, —CF₂O—, —OCF₂—, —CF₂CF₂—, —CH═CH—COO—, —CH═CH—OCO—, —COO—CH═CH—, —OCO—CH═CH—, —CH═CH—, —C≡C—, a single bond, a 1,4-phenylene group, or a trans-1,4-cyclohexylene group, in which an arbitrary hydrogen atom in the 1,4-phenylene group may be substituted with a fluorine atom, and l represents an integer from 2 to 6).

In Formula (II-1) above, M^{y} is preferably a hydrocarbon having from 1 to 10 carbon atoms (one or two or more —CH₂— in the hydrocarbon may be substituted with —O—, —CO—, —COO—, or —OCO— so as not to be directly adjacent to an oxygen atom). In addition, X^{y} is preferably an alkylene group having from 2 to 10 carbon atoms (one or two or more —CH₂— in the alkylene group may be substituted with —O—, —CO—, —COO—, or —OCO— so as not to be directly adjacent to an oxygen atom), a single bond, a 1,4-phenylene group, or a trans-1,4-cyclohexylene group. In addition, 1 number of X^{y}'s in Formula (II-1) above may be the same as or different from one another, but they are preferably the same as one another. l is preferably an integer 2 or more and 4 or less, and it is more preferably 2.

The compound represented by Formula (II-1) according to the present invention is preferably a compound represented by the following Formula (II-2).

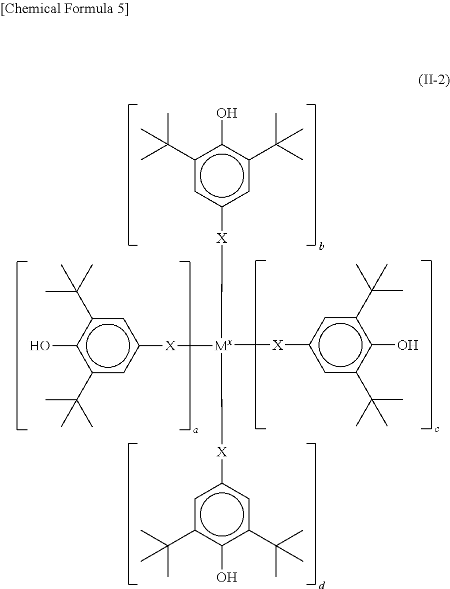

In Formula (II-2) above, M^{x} represents a hydrocarbon having from 1 to 25 carbon atoms (one or two or more —CH₂— in the hydrocarbon may be substituted with —O—, —CO—, —COO—, or —OCO— so as not to be directly adjacent to an oxygen atom), but it is preferably a hydrocarbon having from 2 to 15 carbon atoms (one or two or more —CH₂— in the hydrocarbon may be substituted with —O—, —CO—, —COO—, or —OCO— so as not to be directly adjacent to an oxygen atom).

In Formula (II-2) above, X's each independently represent an alkylene group having from 1 to 15 carbon atoms (one or two or more —CH₂— in the alkylene group may be substituted with —O—, —CO—, —COO—, or —OCO— so as not to be directly adjacent to an oxygen atom), —OCH₂—, —CH₂O—, —COO—, —OCO—, —CF₂O—, —OCF₂—, —CF₂CF₂—, —CH═CH—COO—, —CH═CH—OCO—, —COO—CH═CH—, —OCO—CH═CH—, —CH═CH—, —C≡C—, a single bond, a 1,4-phenylene group, or a trans-1,4-cyclohexylene group (an arbitrary hydrogen atom in the 1,4-phenylene group may be substituted with a fluorine atom), but they are preferably an alkylene group having from 2 to 15 carbon atoms (one or two or more —CH₂— in the alkylene group may be substituted with —O—, —CO—, —COO—, or —OCO— so as not to be directly adjacent to an oxygen atom), a single bond, a 1,4-phenylene group, or a trans-1,4-cyclohexylene group. In addition, four X's in Formula (II-2) above may be the same as or different from one another, but they are preferably the same as one another. l is preferably an integer 2 or more and 4 or less, and it is more preferably 2.

In Formula (II-2) above, a, b, c, and d each independently represent 0 or 1, but a+b+c+d represents 2 or more. a+b+c+d is even more preferably from 2 to 4, and it is even more preferably 2.

The antioxidant represented by Formula (II-1) according to the present invention is preferably a hindered phenol derivative represented by the following Formula (II).

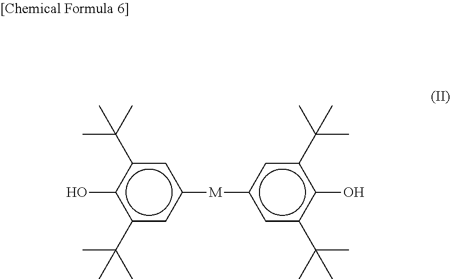

In Formula (II) above, M represents an alkylene group having from 1 to 15 carbon atoms (one or two or more —CH₂— in the alkylene group may be substituted with —O—, —CO—, —COO—, or —OCO— so as not to be directly adjacent to an oxygen atom), —OCH₂—, —CH₂O—, —COO—, —OCO—, —CF₂O—, —OCF₂—, —CF₂CF₂—, —CH═CH—COO—, —CH═CH—OCO—, —COO—CH═CH—, —OCO—CH═CH—, —CH═CH—, —C≡C—, a single bond, a 1,4-phenylene group, or a trans-1,4-cyclohexylene group (an arbitrary hydrogen atom in the 1,4-phenylene group may be substituted with a fluorine atom), but it is preferably an alkylene group having from 1 to 14 carbon atoms, and M in Formula (II) according to the present invention has even more preferably from 2 to 12 carbon atoms, even more preferably from 3 to 10 carbon atoms, even more preferably from 4 to 10 carbon atoms, even more preferably from 5 to 10 carbon atoms, and even more preferably from 6 to 10 carbon atoms since the number of carbon atoms is preferably a great number in consideration of the volatility, but the number of carbon atoms is preferably not too great in consideration of the viscosity.

The antioxidant according to the present invention is even more preferably a compound represented by the following Formula (II-a6), Formula (II-a7), Formula (II-a8), Formula (II-a9), or Formula (II-a10).

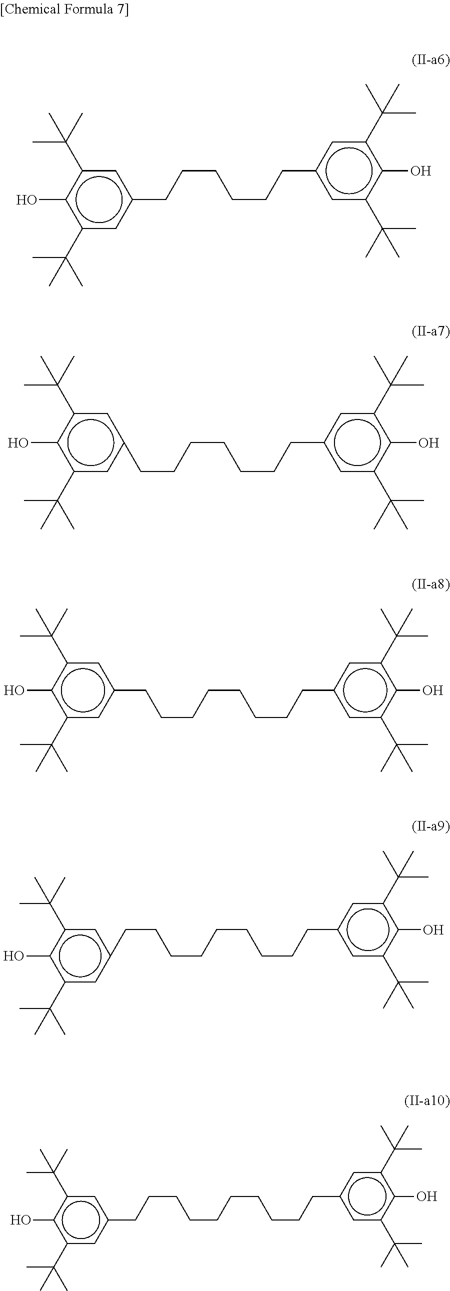

The antioxidant according to the present invention preferably coexists with a liquid crystal compound and more preferably coexists with a nematic liquid crystal compound (or nematic liquid crystal composition). In this case, the liquid crystal composition preferably contains one or more kinds of antioxidants, and the content of the antioxidant is preferably from 0.001 to 1% by mass, more preferably from 0.001 0.5% by mass, and even more preferably from 0.01 to 0.3% by mass.

In addition, the liquid crystal composition according to the present invention contains one kind or two or more kinds of liquid crystal compounds having a positive dielectric anisotropy of 4 or more as the absolute value, and the content of the liquid crystal compound is 3% by mass or more, but it is preferably 5% by mass or more, even more preferably 10% by mass or more, and even more preferably 20% by mass or more. More specifically, it is preferably from 3 to 70% by mass, even more preferably from 5 to 60% by mass, even more preferably from 10 to 60% by mass, and even more preferably from 20 to 60% by mass. Incidentally, the dielectric anisotropy of the liquid crystal compound indicates the value at 20° C. In addition, the term “liquid crystal composition” in the present description is a composition which contains a liquid crystal compound having a positive dielectric anisotropy of 4 or more as the absolute value and the antioxidant according to the present invention.

It is preferable that the liquid crystal composition according to the present invention contains the antioxidant (including compounds represented by Formula (II-1), Formula (II-2), and Formula (II)) according to the present invention and one kind or two or more kinds of compounds represented by Formula (N1):

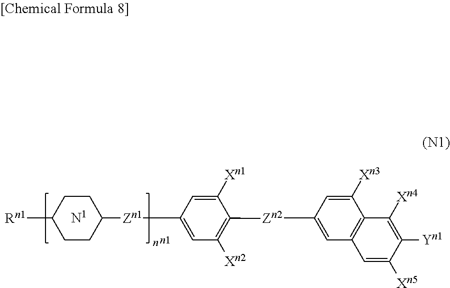

(In Formula (N1) above, Rⁿ¹ represents an alkyl group having from 1 to 10 carbon atoms or an alkenyl group having from 2 to 10 carbon atoms, in which one or non-adjacent two or more —CH₂-in the groups may be each independently substituted with —C≡C—, —O—, —CO—, —COO—, or —OCO— and one or non-adjacent two or more hydrogen atoms in the groups may be each independently substituted with a fluorine atom,

Ring N¹ represents a 1,4-cyclohexylene group, (one or non-adjacent two or more —CH₂— in the group may be substituted with —O— or —S—) or a 1,4-phenylene group (one or non-adjacent two or more —CH═ in the group may be substituted with —N═), in which the hydrogen atoms in the groups may be each independently substituted with a cyano group or a fluorine atom,

Zⁿ¹ and Zⁿ² each independently represent a single bond, —CH₂CH₂—, —(CH₂)₄—, —OCH₂—, —CH₂O—, —OCF₂—, —CF₂O—, —CH₂CH₂CF₂O—, —COO—, —OCO—, or —C≡C—,

nⁿ¹'s each independently represent an integer from 0 to 4, Rings N¹ may be the same as or different from one another when nⁿ¹ is 2 or more, and Zⁿ¹'s may be the same as or different from one another when nⁿ¹ is 1 or more,

Xⁿ¹, Xⁿ², Xⁿ³, Xⁿ⁴, and Xⁿ⁵ each independently represent a hydrogen atom or a fluorine atom, and

Yⁿ¹ 's each independently represent a fluorine atom, a cyano group, a trifluoromethyl group, a fluoromethoxy group, a difluoromethoxy group, a trifluoromethoxy group, or a 2,2,2-trifluoroethyl group but represent a fluorine atom, a trifluoromethyl group, a fluoromethoxy group, a difluoromethoxy group, or a trifluoromethoxy group).

By the combination of a specific liquid crystal compound with a specific antioxidant, it is possible to provide a liquid crystal composition which has suppressed display defects such as a drop mark or a linear residual image and has a low viscosity or a low rotational viscosity, low temperature solubility or a high specific resistance or a high voltage holding ratio, and excellent light resistance or heat resistance.

A liquid crystal composition containing the antioxidant and the compound represented by Formula (N1) contained in the liquid crystal composition of the present invention is preferable from the viewpoint of maintaining a high refractive index anisotropy (Δn) and exhibiting excellent reliability.

In addition, the compound represented by Formula (N1) contained in the liquid crystal composition according to the present invention is preferably a liquid crystal compound having a positive dielectric anisotropy of 4 or more as the absolute value, more preferably a liquid crystal compound having a positive dielectric anisotropy of 5 or more as the absolute value, more preferably a liquid crystal compound having a positive dielectric anisotropy of 6 or more as the absolute value, more preferably a liquid crystal compound having a positive dielectric anisotropy of 7 or more as the absolute value, more preferably a liquid crystal compound having a positive dielectric anisotropy of 8 or more as the absolute value, more preferably a liquid crystal compound having a positive dielectric anisotropy of 9 or more as the absolute value, more preferably a liquid crystal compound having a positive dielectric anisotropy of 10 or more as the absolute value, more preferably a liquid crystal compound having a positive dielectric anisotropy of 15 or more as the absolute value, more preferably a liquid crystal compound having a positive dielectric anisotropy of 20 or more as the absolute value, and more preferably a liquid crystal compound having a positive dielectric anisotropy of 25 or more as the absolute value, and the upper limit of the positive dielectric anisotropy is preferably 50 or less, even more preferably 45 or less, and even more preferably 40 or less.

The content of the compound represented by Formula (N1) in the liquid crystal composition according to the present invention is preferably from 1 to 50% by mass, more preferably from 2 to 45% by mass, even more preferably from 3 to 40% by mass, even more preferably from 5 to 35% by mass, and even more preferably from 10 to 30% by mass.

In the liquid crystal composition according to the present invention, one kind or two or more kinds, preferably from 1 to 10 kinds, more preferably from 1 to 9 kinds, even more preferably from 1 to 8 kinds, and even more preferably from 1 to 7 kinds of compounds represented by Formula (N1) are contained.

The compound represented by Formula (N1) according to the present invention is preferably a compound selected from the group consisting of compounds represented by Formulas (N1-1) and (N1-2), and it is more preferably a compound represented by Formula (N1-2).

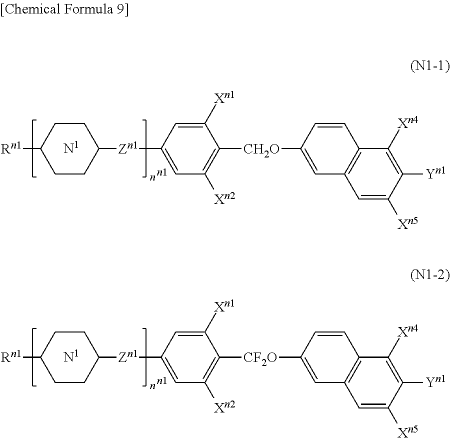

(In Formula (N1-1) and Formula (N1-2) above, Rⁿ¹, Xⁿ¹, Xⁿ², Xⁿ³, Xⁿ⁴, Xⁿ⁵, Yⁿ¹, nⁿ¹, and Zⁿ¹ are as described above, and Ring N¹ represent a 1,4-cyclohexylene group, a tetrahydropyran-2,5-diyl group, a 1,4-phenylene group, a 3-fluoro-1,4-phenylene group, a 3,5-difluoro-1,4-phenylene group, or a dioxane group.) The content of the compound represented by Formula (N1-1) in the liquid crystal composition according to the present invention is preferably from 1 to 50% by mass, more preferably from 2 to 45% by mass, even more preferably from 3 to 40% by mass, even more preferably from 4 to 35% by mass, and even more preferably from 10 to 30% by mass.

The content of the compound represented by Formula (N1-2) in the liquid crystal composition according to the present invention is preferably from 1 to 50% by mass, more preferably from 2 to 45% by mass, even more preferably from 3 to 40% by mass, even more preferably from 4 to 35% by mass, and even more preferably from 10 to 30% by mass.

The compound represented by Formula (N1) according to the present invention is more preferably the following Formula (N3).

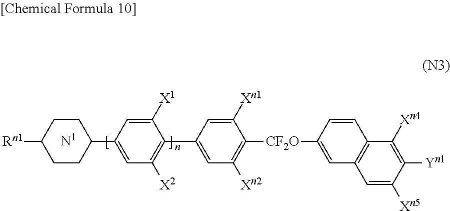

(In Formula (N3) above, Rⁿ¹, Xⁿ¹, Xⁿ², Xⁿ⁴, Xⁿ⁵, and Yⁿ¹ are as described above, X¹ and X² each independently represent a hydrogen atom or a fluorine atom,

Ring N¹ represent a 1,4-cyclohexylene group, a tetrahydropyran-2,5-diyl group, a 1,4-phenylene group, a 3-fluoro-1,4-phenylene group, a 3,5-difluoro-1,4-phenylene group, or a dioxane group, and n is an integer 0 or more and 2 or less.)

The content of the compound represented by Formula (N3) in the liquid crystal composition according to the present invention is preferably from 1 to 50% by mass, more preferably from 2 to 45% by mass, even more preferably from 3 to 40% by mass, even more preferably from 4 to 35% by mass, and even more preferably from 10 to 30% by mass.

As the compound represented by Formula (N3) according to the present invention, the following (N3-1) to (N3-) are preferable.

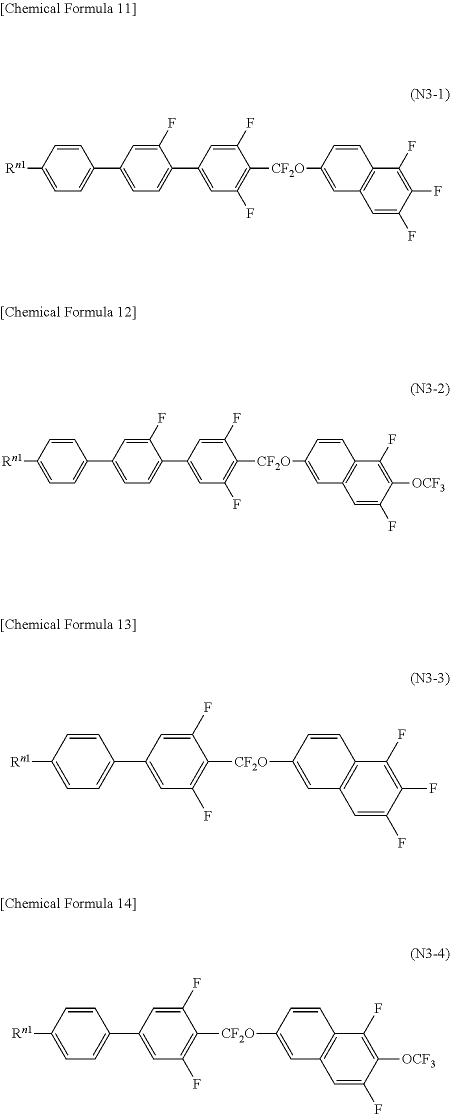

The content of each of the compounds represented by Formula (N3-1) to Formula (N3-4) in the liquid crystal composition according to the present invention is preferably from 0.01 to 15% by mass, more preferably from 0.1 to 12% by mass, and even more preferably from 0.5 to 10% by mass.

The liquid crystal composition according to the present invention preferably contains a compound represented by Formula (K).

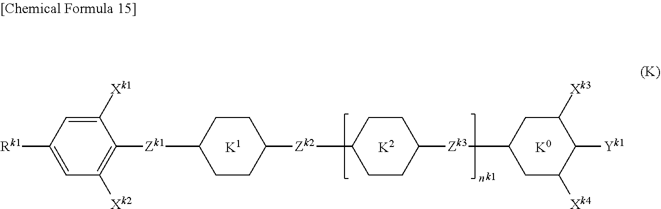

(In Formula (K) above, R^{k1}'s each independently represent an alkyl group having from 1 to 10 carbon atoms or an alkenyl group having from 2 to 10 carbon atoms, in which one or non-adjacent two or more —CH₂— in the groups may be each independently substituted with —C≡C—, —O—, —CO—, —COO—, or —OCO— and one or non-adjacent two or more hydrogen atoms in the groups may be each independently substituted with a fluorine atom,

Ring K¹ and Ring K² each independently represent a 1,4-cyclohexylene group (one or non-adjacent two or more —CH₂-in the group may be substituted with —O— or —S—) or a 1,4-phenylene group (one or non-adjacent two or more —CH═ in the group may be substituted with —N═), in which the hydrogen atoms in the groups may be each independently substituted with a cyano group, a fluorine atom, or a chlorine atom,

Ring K⁰ represents a naphthalene-2,6-diyl group or a 1,4-phenylene group, in which the hydrogen atoms in the groups may be each independently substituted with a fluorine atom,

Z^{k1}, Z^{k2}, and Z^{k3} each independently represent a single bond, —CH₂CH₂—, —(CH₂)₄—, —OCH₂—, —CH₂O—, —OCF₂—, —CF₂O—, —CH₂CH₂CF₂O—, —COO—, —OCO—, or —C≡C—, but they are even more preferably a single bond, —CH₂CH₂—, —OCH₂—, —CH₂O—, —OCF₂—, or —CF₂O—.

n^{k1}'s each independently represent 0, 1, 2, 3, or 4, and Rings K² may be the same as or different from one another when n^{k1} is 2 or more,

X^{k1}, X^{k2}, X^{k3}, and X^{k4} each independently represent a hydrogen atom or a fluorine atom, and Y^{k1} represents a fluorine atom, a cyano group, a trifluoromethyl group, a fluoromethoxy group, a difluoromethoxy group, a trifluoromethoxy group, or a 2,2,2-trifluoroethyl group but it is a fluorine atom, a trifluoromethyl group, a fluoromethoxy group, a difluoromethoxy group, or a trifluoromethoxy group.)

The content of the compound represented by Formula (K) in the liquid crystal composition according to the present invention is preferably from 0 to 30% by mass, more preferably from 1 to 20% by mass, even more preferably from 3 to 20% by mass, and even more preferably from 3 to 15% by mass.

In the liquid crystal composition according to the present invention, one kind or two or more kinds, preferably from 1 to 10 kinds, more preferably from 1 to 8 kinds, even more preferably from 1 to 6 kinds, and even more preferably from 1 to 3 kinds are contained in the case of adding the compounds represented by Formula (K).

The compound represented by Formula (K) above is preferably a compound represented by the following Formula (K1).

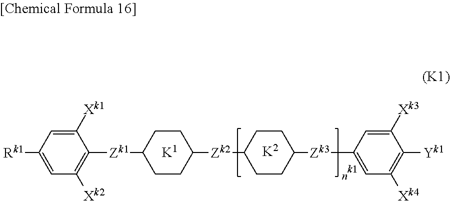

(In Formula (K1) above, R^{k1}, Ring K¹, Ring K², n^{k1}, X^{k1}, X^{k2}, X^{k3}, X^{k4}, Y^{k1}, Z^{k1}, Z^{k2}, and Z^{k3} are as described in Formula (K) above, and thus the description thereon is omitted here.)

The content of the compound represented by Formula (K1) in the liquid crystal composition according to the present invention is preferably from 0 to 30% by mass, more preferably from 1 to 20% by mass, even more preferably from 3 to 20% by mass, and even more preferably from 3 to 15% by mass.

The compound represented by Formula (K1) according to the present invention is preferably a compound selected from the group of compounds represented by the following Formulas (K11) to (K24), and it is even more preferably a compound selected from the group of compounds represented by Formulas (K11) to (K17).

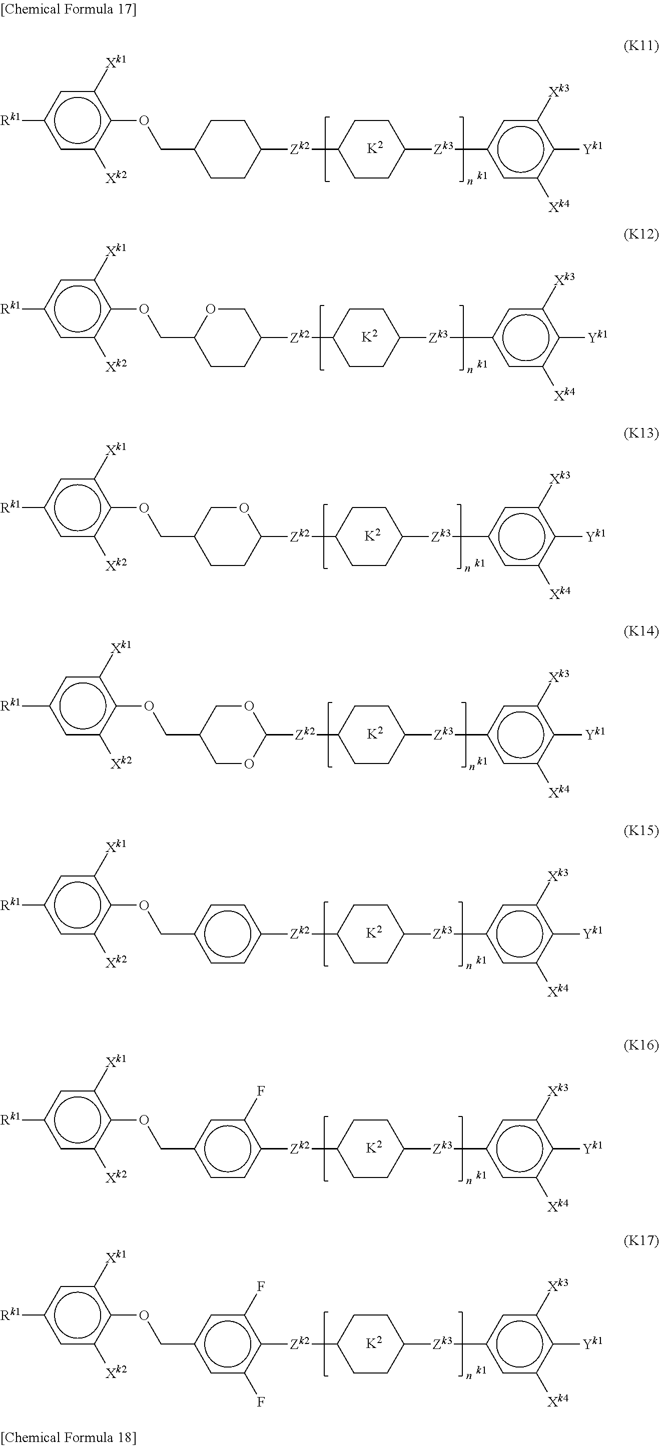

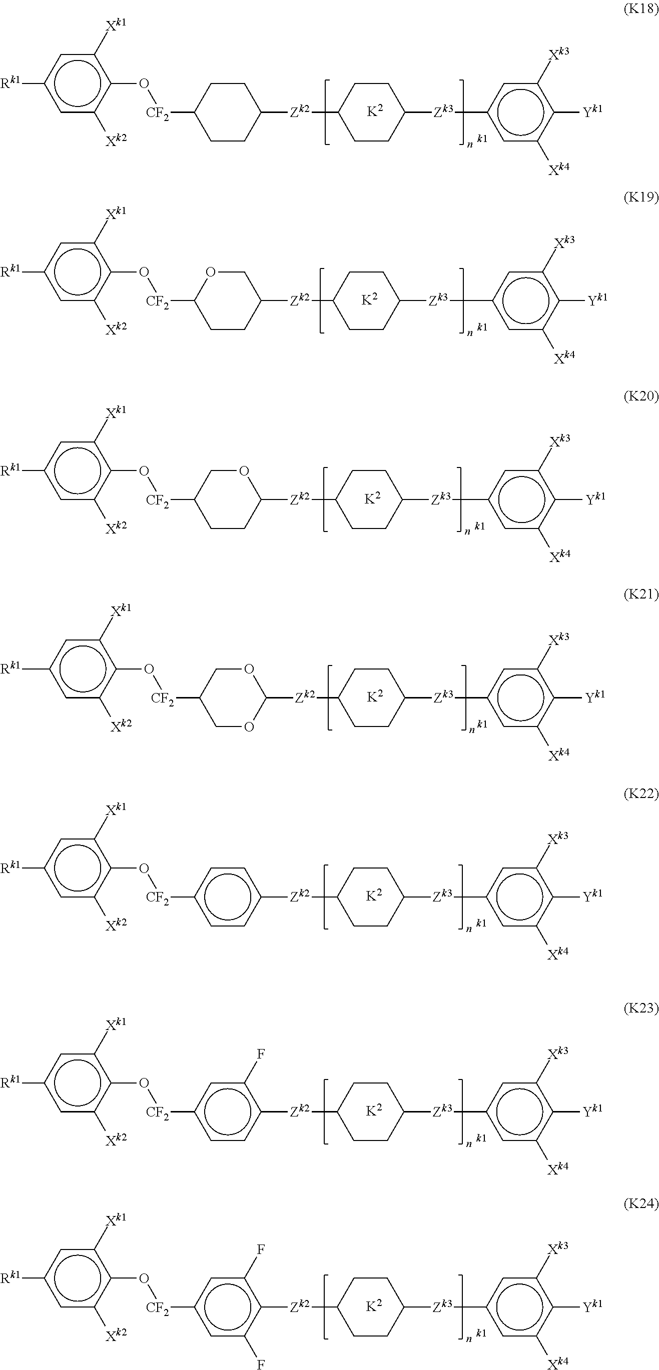

(In Formulas (K11) to (K24) above, R^{k1}, Ring K², Z^{k2}, Z^{k3}, n^{k1}, X^{k1}, X^{k2}, X^{k3}, X^{k4}, and Y^{k1} are as described above.)

The compound represented by Formula (K) above is preferably a compound represented by the following Formula (K2).

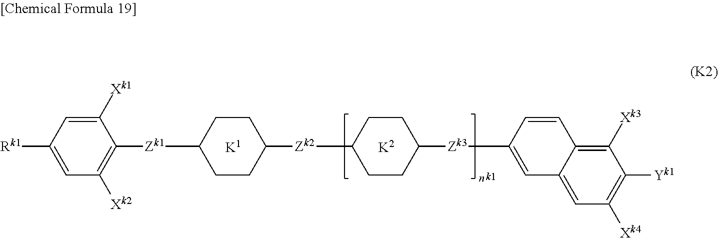

(In Formula (K2) above, R^{k1}, Ring K¹, Ring K², n^{k1}, X^{k1}, X^{k2}, X^{k3}, X^{k4}, Y^{k1}, Z^{k1}, Z^{k2}, and Z^{k3} are as described in Formula (K) above, and thus the description thereon is omitted here.)

The content of the compound represented by Formula (K2) in the liquid crystal composition according to the present invention is preferably from 0 to 30% by mass, more preferably from 1 to 20% by mass, even more preferably from 3 to 20% by mass, and even more preferably from 3 to 15% by mass.

The compound represented by Formula (K2) according to the present invention is preferably a compound selected from the group of compounds represented by the following Formulas (K25) to (K28), and it is even more preferably a compound selected from the group of compounds represented by Formulas (K25) and (K26).

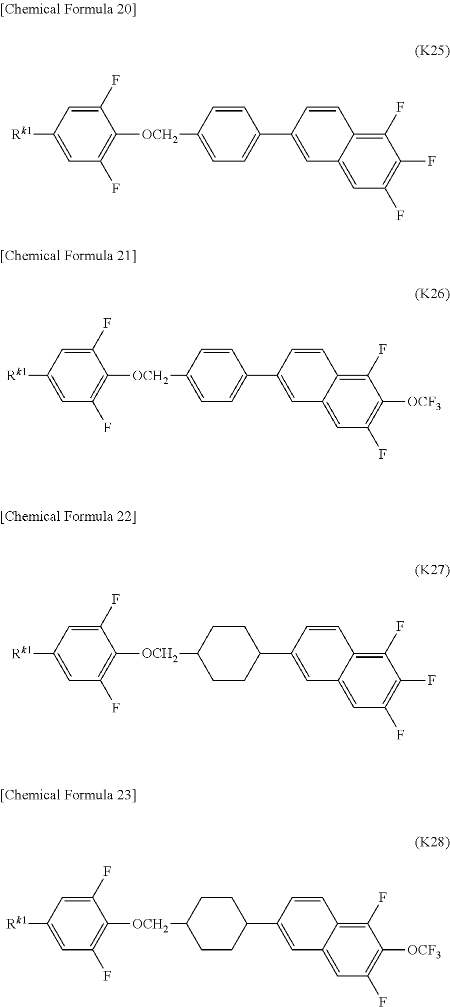

The content of each of the compounds represented by Formulas (K25) to (K28) in the liquid crystal composition according to the present invention is preferably from 0 to 15% by mass, more preferably from 0.01 to 12% by mass, and even more preferably from 0.5 to 10% by mass.

The liquid crystal compound which has a positive dielectric anisotropy of 4 or more as the absolute value and is contained in the liquid crystal composition of the present invention is also preferably Formula (Pa).

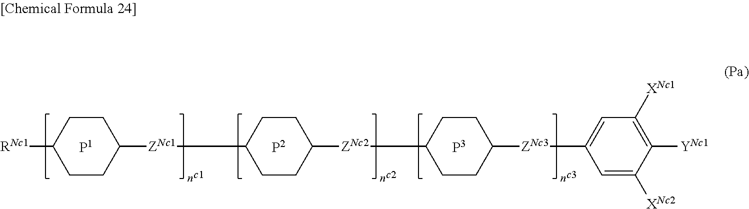

In Formula (Pa) above, R^{Nc1} represents an alkyl group having from 1 to 8 carbon atoms or an alkenyl group having from 2 to 8 carbon atoms, in which one or non-adjacent two or more —CH₂-in the groups may be each independently substituted with —C≡C—, —O—, —CO—, —COO—, or —OCO—, but it is preferably an alkyl group having from 1 to 5 carbon atoms or an alkenyl group having from 2 to 5 carbon atoms.

In Formula (Pa) above, Rings P¹, P², and P³ each independently represent a 1,4-cyclohexylene group (one or non-adjacent two or more —CH₂— in the group may be substituted with —O— or —S—) or a 1,4-phenylene group (one or non-adjacent two or more —CH═ in the group may be substituted with —N═), in which the hydrogen atoms in the groups may be each independently substituted with a cyano group, a fluorine atom, or a chlorine atom, but they are preferably a 1,4-cyclohexylene group, a tetrahydropyran-2,5-diyl group, a 1,4-phenylene group, a 3-fluoro-1,4-phenylene group, or a 3, 5-difluoro-1,4-phenylene group.

In Formula (Pa) above, Z^{Nc1}, Z^{Nc2}, and Z^{Nc3} each independently represent a single bond, —CH₂CH₂—, —(CH₂)₄—, —OCH₂—, —CH₂O—, —OCF₂—, —CF₂O—, —COO—, —OCO—, or —C≡C—, but they are even more preferably a single bond, —CH₂CH₂—, —CH₂O—, or —CF₂O—, and any one of existing Z^{Nc1}, Z^{Nc2}, and Z^{Nc3} is even more preferably a single bond.

In Formula (Pa) above, n^{c1}, n^{c2}, and n^{c3} each independently represent 0, 1, or 2. n^{c1}+n^{c2}+n^{c3} represents from 1 to 5, but it is even more preferably 4 or less and even more preferably 3 or less.

In Formula (Pa) above, X^{Nc1} and X^{Nc2} each independently represent a hydrogen atom or a fluorine atom.

In Formula (Pa) above, Y^{Nc1} represents a fluorine atom, a chlorine atom, a cyano group, a trifluoromethyl group, a fluoromethoxy group, a difluoromethoxy group, a trifluoromethoxy group, or a 2,2,2-trifluoroethyl group, but it is preferably a fluorine atom, a trifluoromethyl group, a fluoromethoxy group, a difluoromethoxy group, or a trifluoromethoxy group, and it is even more preferably a fluorine atom, a trifluoromethyl group, or a trifluoromethoxy group.

The content of the compound represented by Formula (Pa) in the liquid crystal composition according to the present invention is preferably from 0 to 50% by mass, more preferably from 1 to 40% by mass, even more preferably from 1.5 to 30% by mass, even more preferably from 3 to 30% by mass, and even more preferably from 5 to 30% by mass.

The compound represented by Formula (Pa) according to the present invention is preferably a compound selected from the group of compounds represented by Formulas (P01) to (P80).

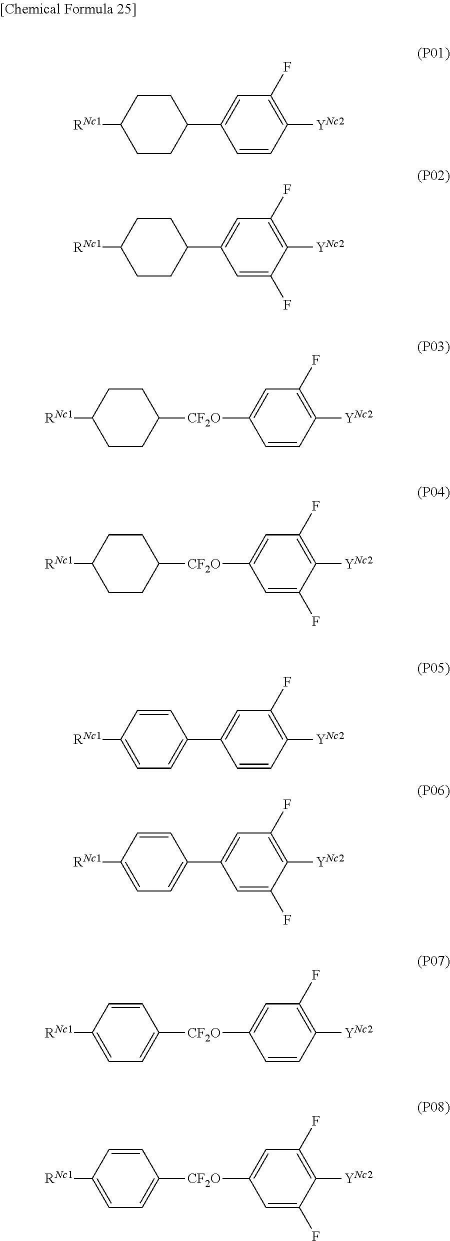

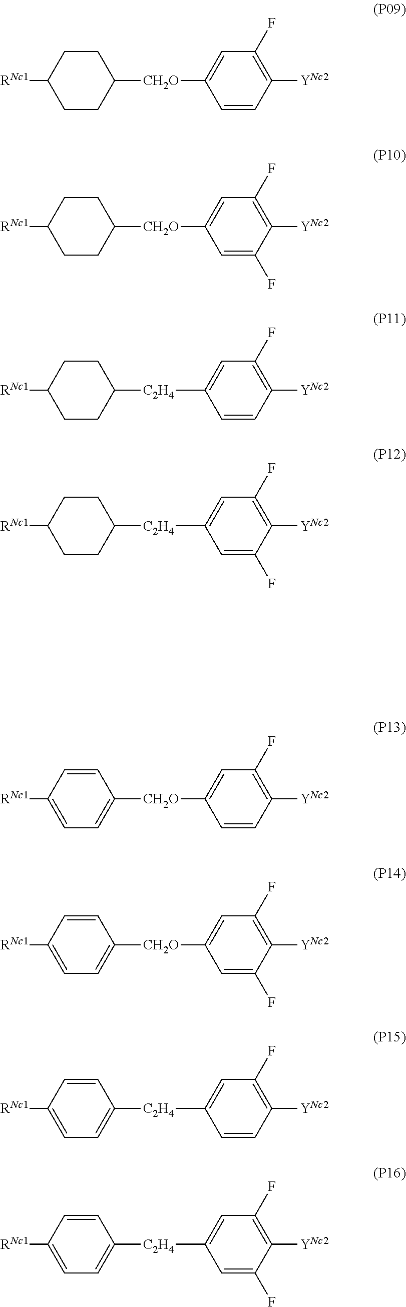

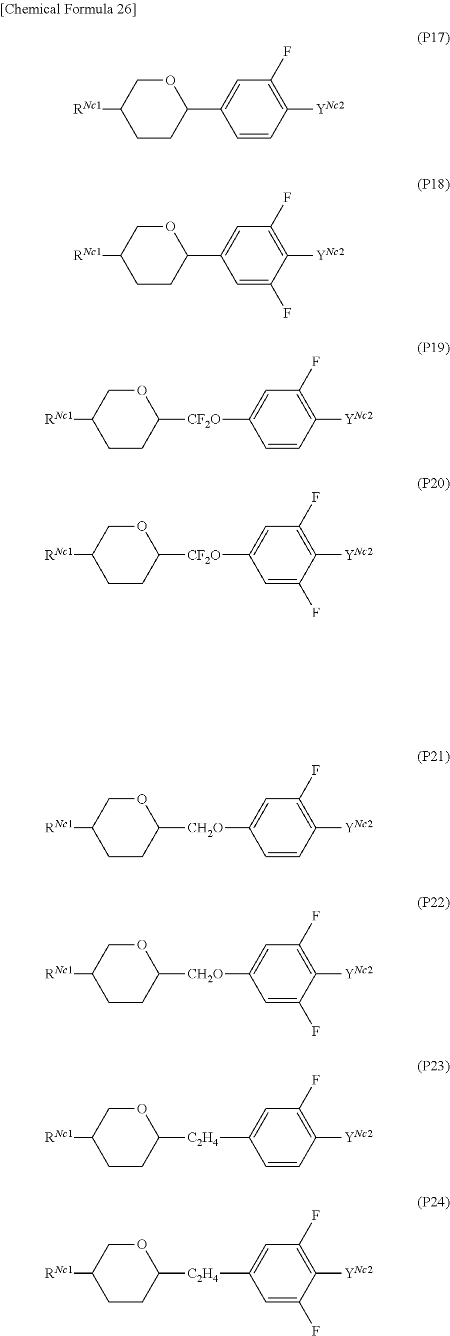

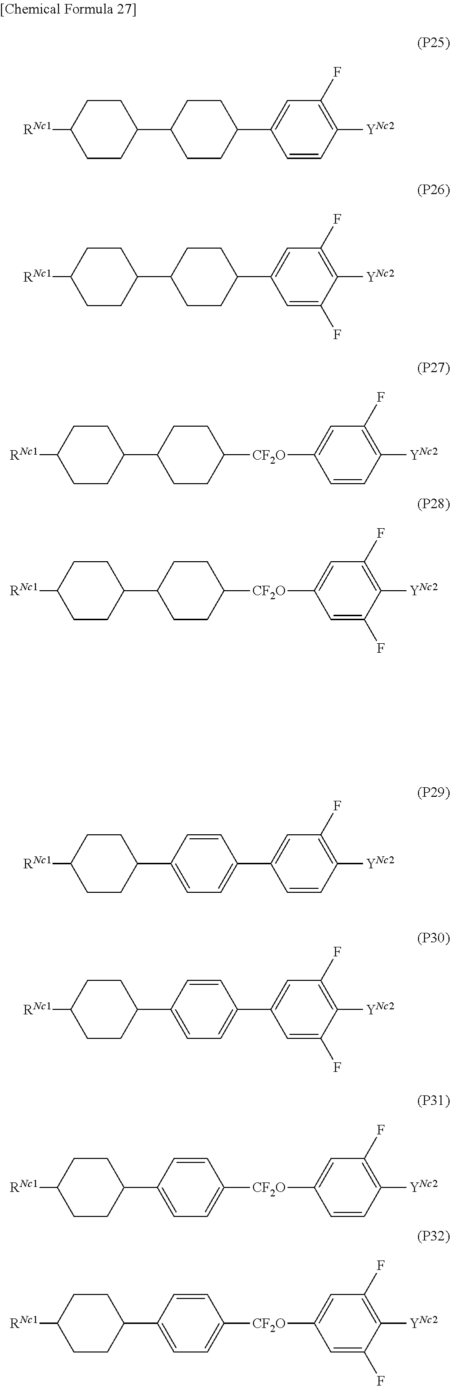

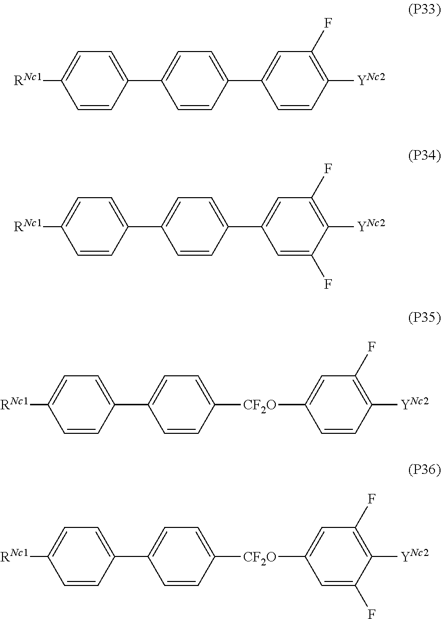

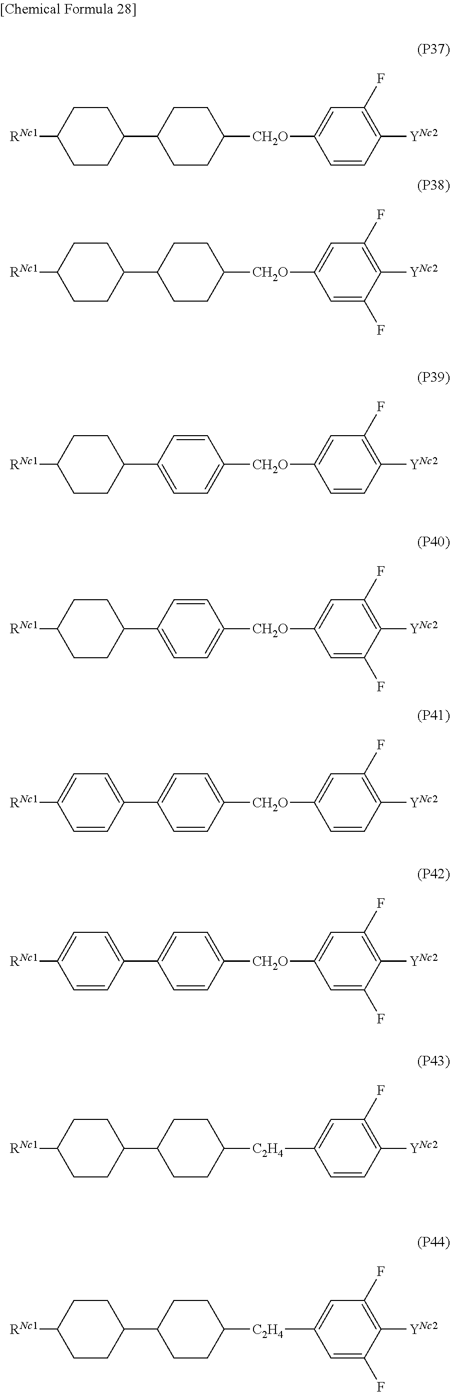

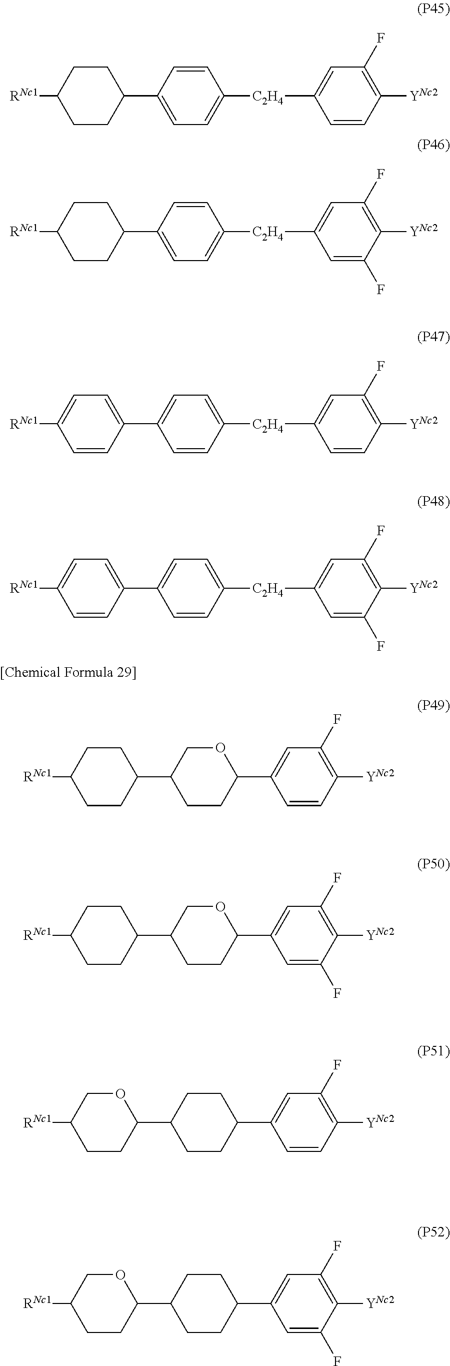

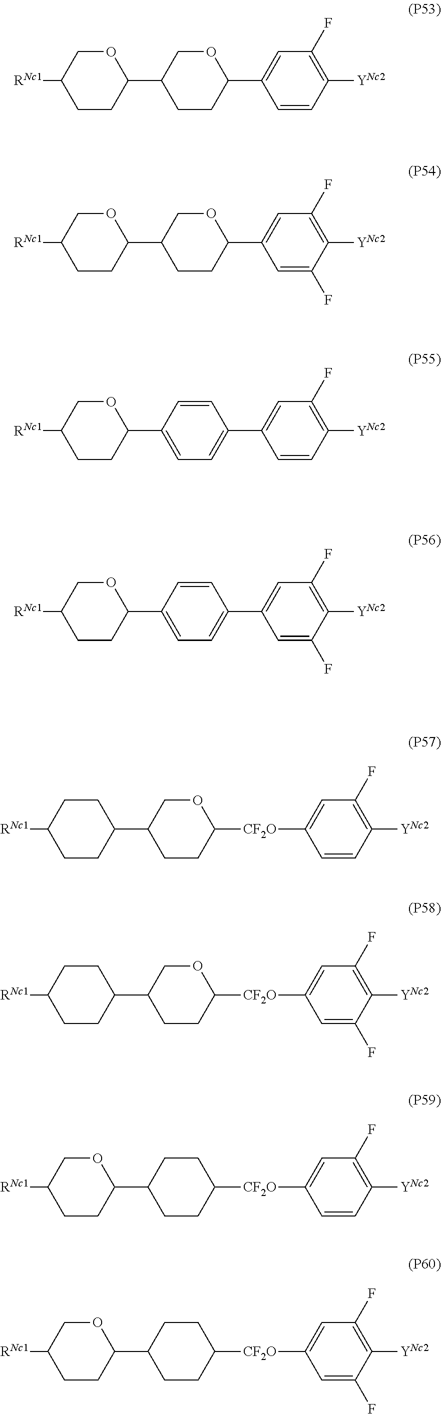

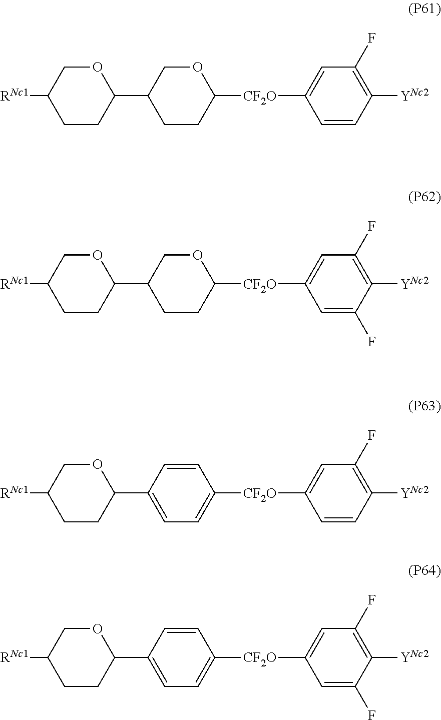

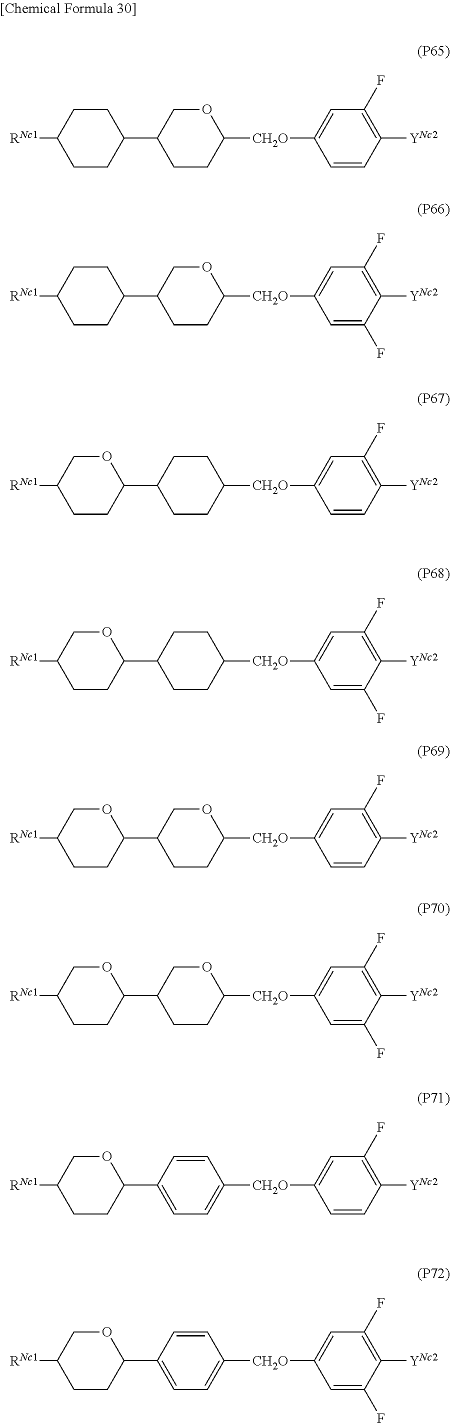

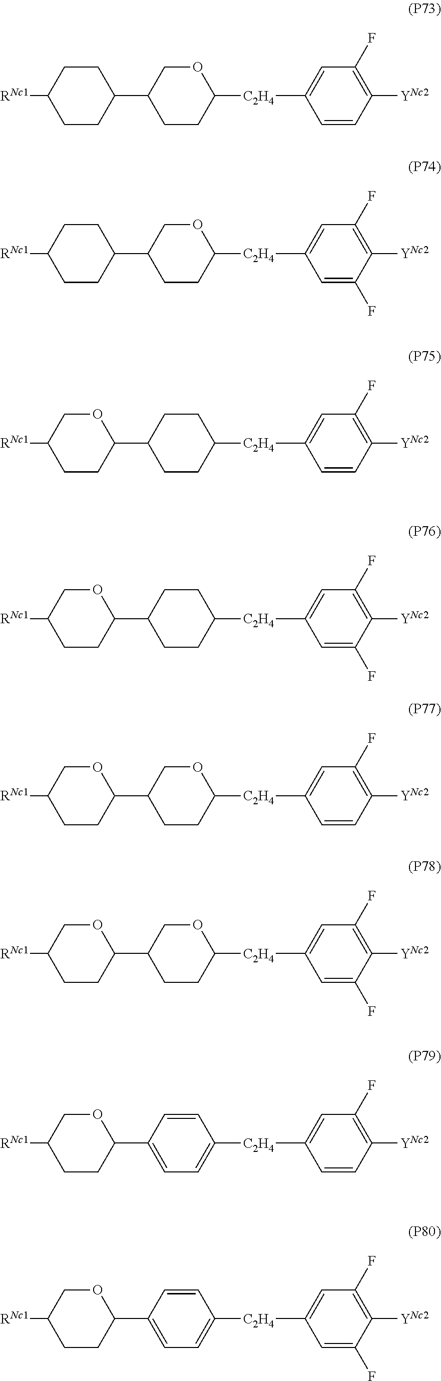

In Formulas (P1) to (P80) above, R^{Nc1} represents an alkyl group having from 1 to 8 carbon atoms or an alkenyl group having from 2 to 8 carbon atoms, in which one or non-adjacent two or more —CH₂— in the groups may be each independently substituted with —C≡C—, —O—, —CO—, —COO—, or —OCO—, but it is preferably an alkyl group having from to 5 carbon atoms or an alkenyl group having from 2 to 5 carbon atoms.

In Formulas (P1) to (P80) above, Y^{Nc2} represents a hydrogen atom, a fluorine atom, a chlorine atom, a cyano group, a trifluoromethyl group, a fluoromethoxy group, a difluoromethoxy group, a trifluoromethoxy group, or a 2,2,2-trifluoroethyl group, but it is preferably a fluorine atom, a trifluoromethyl group, a fluoromethoxy group, a difluoromethoxy group, or a trifluoromethoxy group, and it is even more preferably a fluorine atom, a trifluoromethyl group, or a trifluoromethoxy group.

Examples of the compound represented by Formula (Pa) according to the present invention may further include the following compounds.

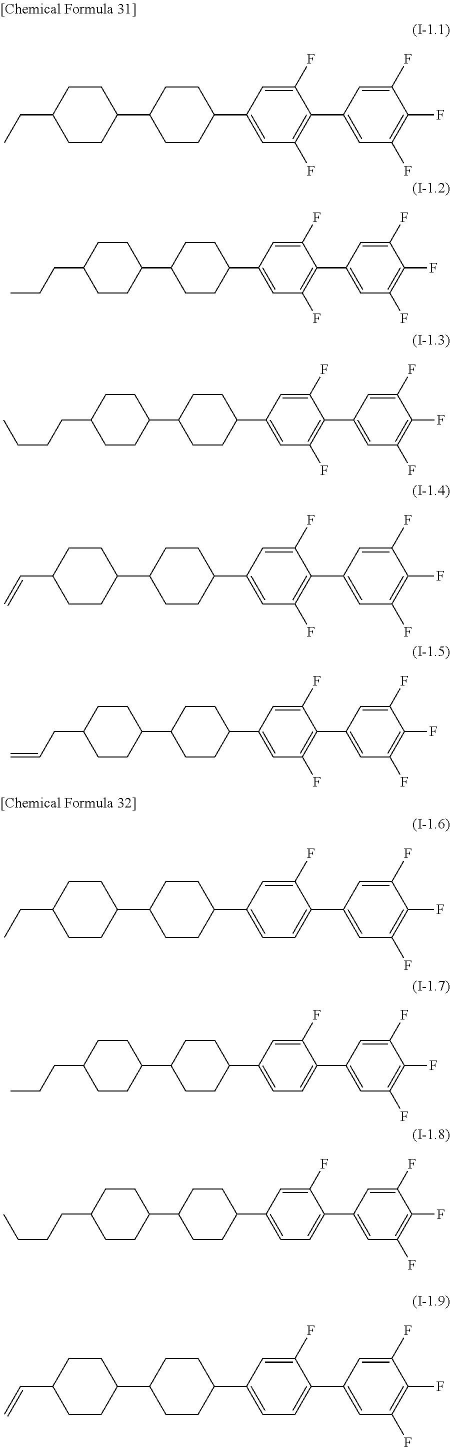

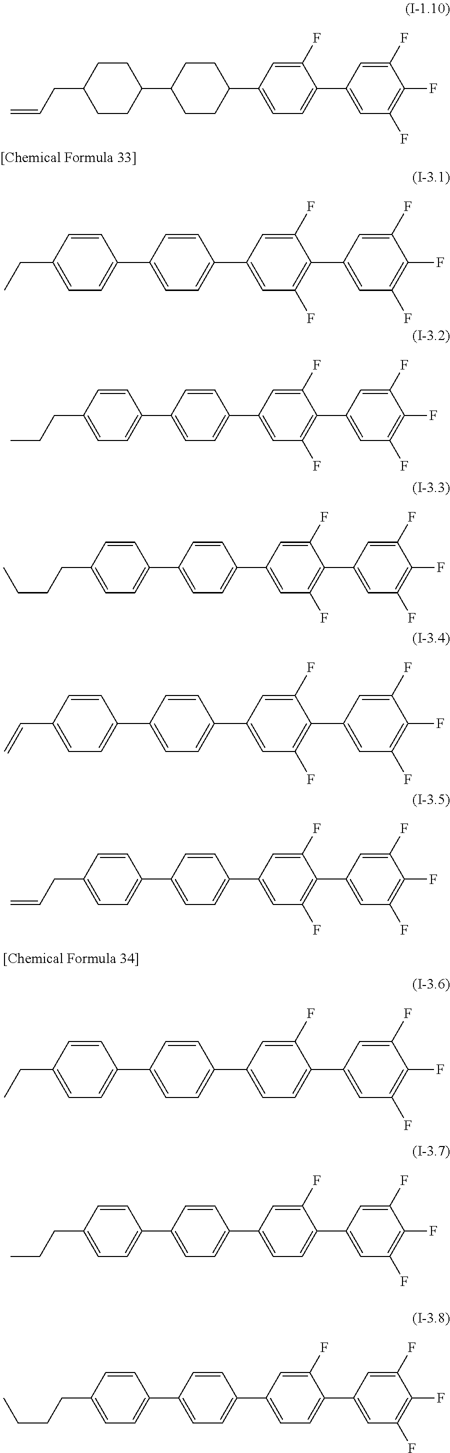

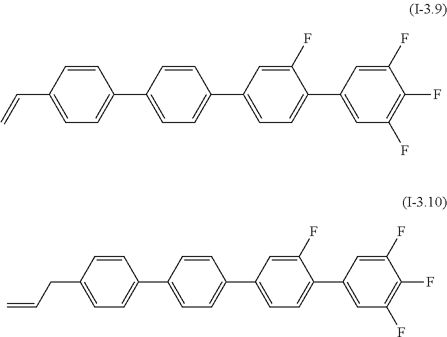 [SMILES: C=Cc1ccc(-c2ccc(-c3ccc(-c4cc(F)c(F)c(F)c4)c(F)c3)cc2)cc1 ; C=CCc1ccc(-c2ccc(-c3ccc(-c4cc(F)c(F)c(F)c4)c(F)c3)cc2)cc1]

The liquid crystal composition according to the present invention can contain a compound having a dielectric anisotropy (Δ∈) of from −1 to +1 (preferably, a dielectric anisotropy (Δ∈) of from −0.5 to +0.5) as a third component other than the antioxidant or the liquid crystal compound having a positive dielectric anisotropy of 4 or more as the absolute value.

As the third component, it is preferable to contain one kind or two or more kinds of compounds represented by the following Formula (L)

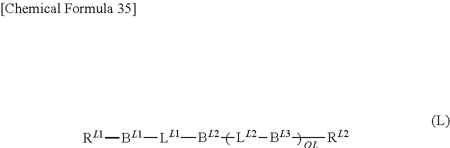

(In Formula (L) above, R^{L1} and R^{L2} each independently represent an alkyl group having from 1 to 10 carbon atoms or an alkenyl group having from 2 to 10 carbon atoms, and one or non-adjacent two or more —CH₂— in the alkyl group may be each independently substituted with —CH═CH—, —C≡C—, —O—, —CO—, —COO—, or —OCO—,

OL represents 0, 1, 2, or 3,

B^{L1}, B^{L2} and B^{L3} each independently represent a group selected from the group consisting of

(a) a 1,4-cyclohexylene group (one —CH₂— or non-adjacent two or more —CH₂— present in this group may be substituted with —O—) and
(b) a 1,4-phenylene group (one —CH═ or non-adjacent two or more —CH═ present in this group may be substituted with —N═), in which one and/or two or more hydrogen atoms in the group (a) and the group (b) above may be each independently substituted with a cyano group, a fluorine atom, or a chlorine atom,

L^{L1} and L^{L2} each independently represent a single bond, —CH₂CH₂—, —(CH₂)₄—, —OCH₂—, —CH₂O—, —COO—, —OCO—, —OCF₂—, —CF₂O—, —CH═N—N═CH—, —CH═CH—, —CF═CF—, or —C≡C—, and

L^{L2}'s may be the same as or different from one another when OL is a 2 or 3 and a plurality of L² are present, and B^{L3}'s may be the same as or different from one another when OL is 2 or 3 and a plurality of B^{L3} are present. However, the compounds represented by Formula (K), Formula (N1), and Formula (Pa) are excluded.), it is more preferable to contain from 1 kind to 20 kinds of compounds represented by Formula (L), and it is even more preferable to contain from 2 kinds to 10 kinds of compounds represented by Formula (L).

The compound represented by Formula (L) above is preferably a compound selected from the group of compounds represented by Formula (III-A) to Formula (III-J).

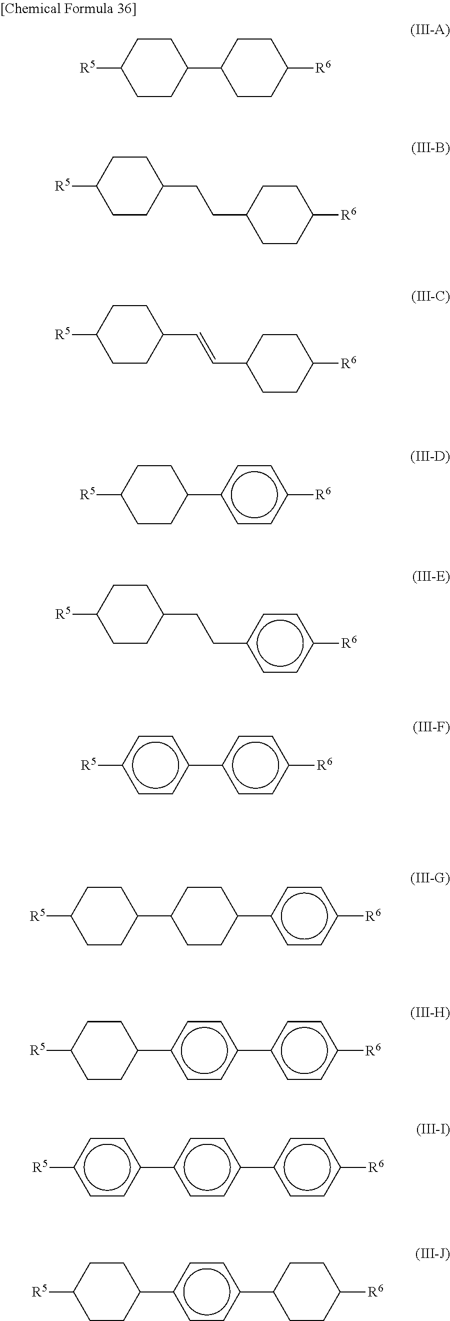

In Formula (III-A) to (III-J) above, R⁵ is preferably an alkyl group having from 1 to 10 carbon atoms or an alkenyl group having from 2 to 10 carbon atoms and more preferably an alkyl group having from 1 to 5 carbon atoms or an alkenyl group having from 2 to 5 carbon atoms. R⁶ is preferably an alkyl group having from 1 to 10 carbon atoms, an alkoxy group having from 1 to 10 carbon atoms, an alkenyl group having from 2 to 10 carbon atoms, or an alkenyloxy group having from 2 to 10 carbon atoms, and it is more preferably an alkyl group having from 1 to 5 carbon atoms, an alkoxy group having from 1 to 5 carbon atoms, an alkenyl group having from 2 to 5 carbon atoms, or an alkenyloxy group having from 2 to 5 carbon atoms.

The third component in the liquid crystal composition according to the present invention is even more preferably a compound selected from Formula (III-A), Formula (III-D), Formula (III-F), Formula (III-G), and Formula (III-H), and it is even more preferably a compound selected from Formula (III-A), Formula (III-F), Formula (III-G), and Formula (III-H).

In addition, in the compounds represented by Formula (III-D), Formula (III-G), and Formula (III-H), R⁵ is preferably an alkyl group having from 1 to 5 carbon atoms or an alkenyl group having from 2 to 5 carbon atoms, and R⁶ is preferably an alkyl group having from 1 to 5 carbon atoms or an alkoxy group having from 1 to 5 carbon atoms. In the compound represented by Formula (III-F), R⁵ and R⁶ are each independently preferably an alkyl group having from 1 to 5 carbon atoms or an alkenyl group having from 2 to 5 carbon atoms.

The content of the compound represented by Formula (L) that is the third component is preferably from 0 to 20% by mass, more preferably from 1 to 20% by mass, even more preferably from 3 to 20% by mass, and even more preferably from 3 to 15% by mass.

The liquid crystal composition may contain one kind or two or more kinds of compounds represented by Formula (VIII-c) to Formula (VIII-e) as the compound represented by Formula (L) above.

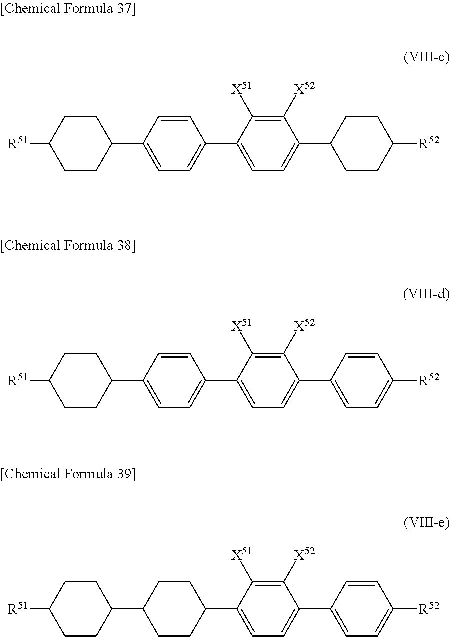

In Formula (VIII-c) to Formula (VIII-e), R⁵¹ and R⁵² each independently represent an alkyl group having from 1 to 5 carbon atoms, an alkoxy group having from 1 to 5 carbon atoms, an alkenyl group having from 2 to 5 carbon atoms, an alkenyloxy group having from 2 to 5 carbon atoms, and X⁵¹ and X⁵² each independently represent a fluorine atom, a chlorine atom, or a hydrogen atom.

More specifically, a compound of Formula (V-6.1) is mentioned.

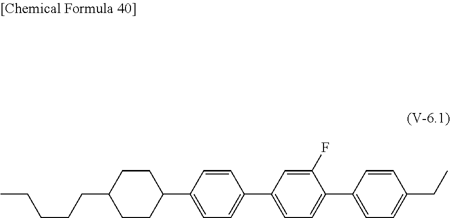 [SMILES: CCCCCC1CCC(c2ccc(-c3ccc(-c4ccc(CC)cc4)c(F)c3)cc2)CC1]

The liquid crystal composition may contain one kind or two or more kinds of compounds represented by Formula (V-9.1) to Formula (V-9.3) as the compound represented by Formula (L) above.

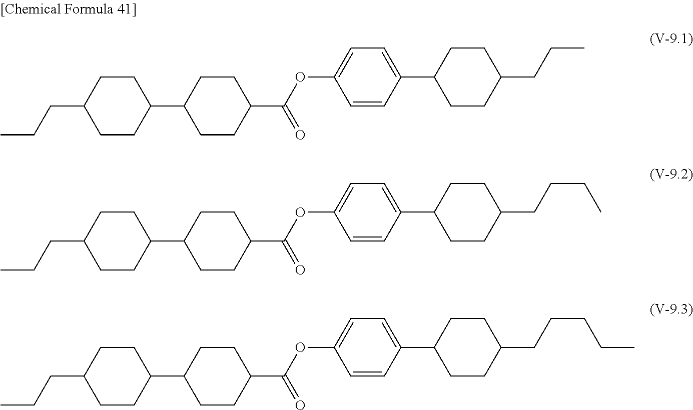

An embodiment of the liquid crystal composition according to the present invention preferably contains an antioxidant represented by Formula (II-1), a compound represented by Formula (N1), and a compound represented by Formula (L), it more preferably contains an antioxidant represented by Formula (II-1), a compound represented by Formula (N1), a compound represented by Formula (L), and a compound represented by Formula (Pa), and it more preferably contains an antioxidant represented by Formula (II-1), a compound represented by Formula (N1), a compound represented by Formula (L), a compound represented by Formula (Pa), and a compound represented by Formula (K1). In addition, another embodiment preferably contains an antioxidant represented by Formula (II-1), a compound represented by Formula (N1-2), and a compound represented by Formula (L).

Another preferred embodiment of the liquid crystal composition according to the present invention contains an antioxidant represented by Formula (II-1) and at least one or more kinds of compounds represented by Formula (N1) and/or compounds represented by Formula (K), and more preferred embodiment contains an antioxidant represented by Formula (II-1), at least one or more kinds of compounds represented by Formula (N1) and/or compounds represented by Formula (K), and at least one or more kinds of compounds represented by Formula (L). In addition, another preferred embodiment of the liquid crystal composition according to the present invention contains an antioxidant represented by Formula (II-1), at least one or more kinds of compounds represented by Formula (N1), at least one or more kinds of compounds represented by Formula (K2), and at least one or more kinds of compounds represented by Formula (L).

The liquid crystal composition of the present invention has a dielectric anisotropy (Δ∈) at 20° C. of from 2.0 to 20.0, but the dielectric anisotropy (Δ∈) is preferably from 2.0 to 18.0, even more preferably from 3.0 to 16.0, and even more preferably from 4.0 to 16.0.

The liquid crystal composition of the present invention has a refractive index anisotropy (Δn) at 20° C. of from 0.08 to 0.18, but the refractive index anisotropy (Δn) is even more preferably from 0.09 to 0.15 and even more preferably from 0.09 to 0.12. In more detail, it is preferably from 0.10 to 0.18 in the case of coping with a thin cell gap, and it is preferably from 0.08 to 0.10 in the case of coping with a thick cell gap.

The liquid crystal composition of the present invention has a nematic phase-isotropic liquid phase transition temperature (Tₙᵢ) of from 60° C. to 120° C., but the nematic phase-isotropic liquid phase transition temperature (Tₙᵢ) is more preferably from 70° C. to 110° C. and even more preferably from 70° C. to 100° C.

The liquid crystal composition of the present invention has a viscosity (η) at 20° C. of from 10 to 30 mPa·s, but the viscosity (η) is even more preferably from 10 to 25 mPa·s and even more preferably from 10 to 20 mPa·s.

The liquid crystal composition of the present invention has a rotation viscosity (γ₁) at 20° C. of from 50 to 130 mPa·s, but the rotation viscosity (γ₁) is even more preferably from 50 to 110 mPa·s and even more preferably from 50 to 90 mPa·s.

The liquid crystal composition according to the present invention can contain a polymerizable compound in order to fabricate a liquid crystal display device having a PS mode, a horizontal electric field type PSA mode, or a horizontal electric field type PSVA mode. Examples of the polymerizable compound which can be used may include a biphenyl derivative of which the polymerization proceeds by an energy ray such as light and a photopolymerizable monomer having a liquid crystal backbone formed by the linkage of a plurality of six-membered rings such as a terphenyl derivative. For example, the liquid crystal composition according to the present invention preferably contains a polymerizable compound such as a biphenyl derivative or a terphenyl derivative at from 0.01 to 2% by mass as the polymerizable monomer. In more detail, the liquid crystal composition of the present invention contains one kind or two or more kinds of polymerizable compounds represented by Formula (M).

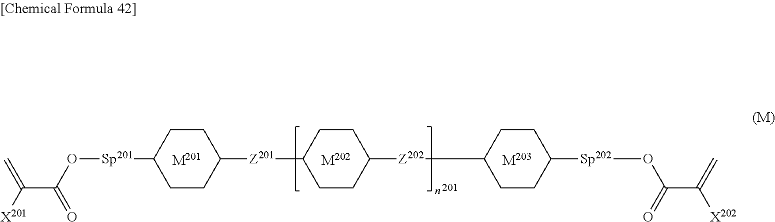

In Formula (M), X²⁰¹ and X²⁰² each independently represent a hydrogen atom, a methyl group, or a —CF₃ group. A diacrylate derivative in which both of X²⁰¹ and X²⁰² are a hydrogen atom and a dimethacrylate derivative both of X²⁰¹ and X²⁰² are a methyl group are preferable, and a compound in which either of X²⁰¹ or X²⁰² is a hydrogen atom and the other is a methyl group is also preferable. It is possible to use a preferred compound depending on the application, but in a PSA display device, it is also preferable that the polymerizable compound represented by Formula (M) has at least one methacrylate derivative and it is also preferable to have two methacrylate derivatives.

Sp²⁰¹ and Sp²⁰² each independently represent a single bond, an alkylene group having from 1 to 8 carbon atoms, or —O—(CH₂)ₛ— (in the formula, s represents an integer from 2 to 7 and the oxygen atom is chemically bonded to a ring). In a liquid crystal display device having a PSA mode, it is preferable that at least either of Sp²⁰¹ or Sp²⁰² is a single bond, and a compound in which both of Sp²⁰¹ and Sp²⁰² are a single bond is preferable, or it is preferable that either of Sp²⁰¹ or Sp²⁰² is a single bond and the other is an alkylene group having from 1 to 8 carbon atoms or —O—(CH₂)ₛ—, and in this case, an alkylene group having from 1 to 4 carbon atoms is preferable and s is preferably from 1 to 4.

Ring M²⁰¹, Ring M²⁰², and Ring M²⁰³ each independently represent a trans-1,4-cyclohexylene group (one or non-adjacent two or more —CH₂— in the group may be substituted with —O— or —S—), a 1,4-phenylene groups (one or non-adjacent two or more —CH═ in the group may be substituted with —N═), a 1,4-cyclohexenylene group, a1,4-bicyclo[2.2.2]octylenegroup, a piperidine-1,4-diyl group, a naphthalene-2,6-diyl group, a decahydronaphthalene-2,6-diyl group, or a 1,2,3,4-tetrahydronaphthalene-2,6-diyl group, in which the hydrogen atoms in the groups may be each independently substituted with a fluorine atom, a —CF₃ group, an alkyl group having from 1 to 10 carbon atoms, an alkoxy group having from 1 to 10 carbon atoms, or any of Formula (R-1) to Formula (R-15).

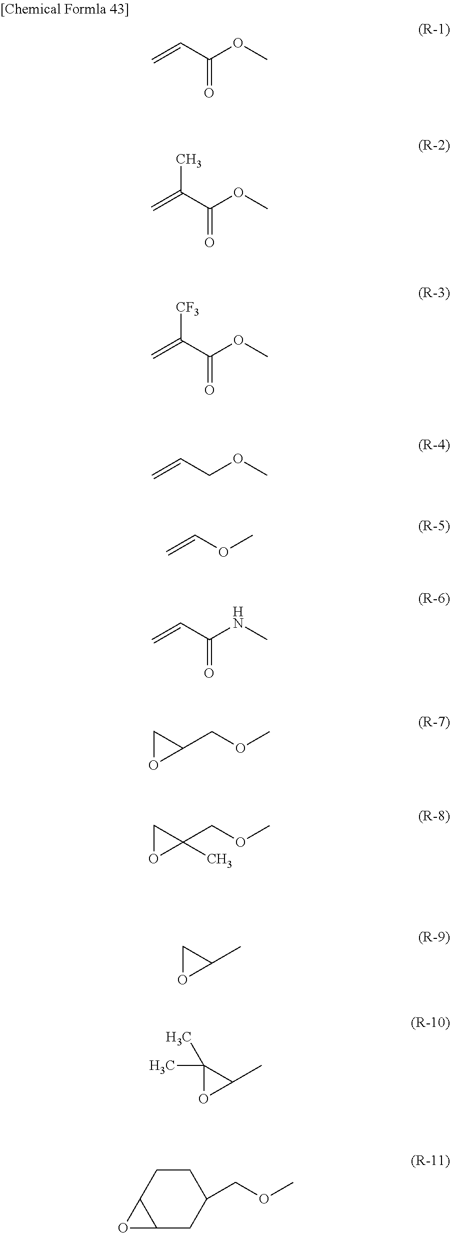

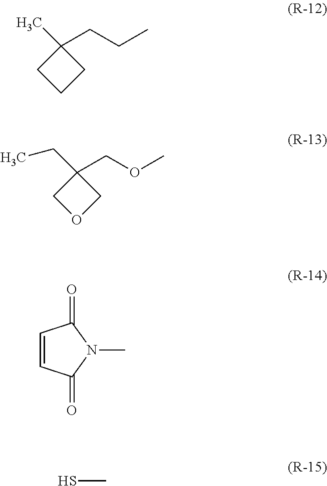

Z²⁰¹ and Z²⁰² each independently represent —OCH₂—, —CH₂O—, —COO—, —OCO—, —CF₂O—, —OCF₂—, —CH₂CH₂—, —CF₂CF₂—, —CH—CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —COO—CH₂—, —OCO—CH—, —CH₂—COO—, —CH₂—OCO—, —CY¹═CY²— (in the formula, Y¹ and Y² each independently represent a fluorine atom or a hydrogen atom), —C≡C—, or a single bond, but they are preferably —COO—, —OCO—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, —C≡C—, or a single bond and even more preferably —COO—, —OCO—, —CH═CH—COO—, —CH═CH—OCO—, —COO—CH═CH—, —OCO—CH═CH—, —COO—CH₂CH₂—, —OCO—CH₂CH₂—, —CH₂CH₂—COO—, —CH₂CH₂—OCO—, or a single bond.

n²⁰¹ represents 0, 1, or 2, but it is preferably 0 or 1. However, a plurality of Rings M²⁰² may be the same as or different from one another and a plurality of Z²⁰²'s may be the same as or different from one another.

The polymerizable compound-containing liquid crystal composition of the present invention contains at least one kind of polymerizable compound represented by Formula (M), but it preferably contains from one kind to five kinds of polymerizable compounds represented by Formula (M), and it even more preferably contains from one kind to three kinds of polymerizable compounds represented by Formula (M). In a case in which the content of the polymerizable compound represented by Formula (M) is small, the alignment regulating force of the polymerizable compound with respect to the liquid crystal composition is weakened. In contrast, in a case in which the content of the polymerizable compound represented by Formula (M) is too great, the energy required at the time of the polymerization increases and the amount of the polymerizable compound that is not polymerized but remains increases to be the factor of display defects, and thus the content of the polymerizable compound is preferably from 0.01 to 2.00% by mass, more preferably from 0.05 to 1.00% by mass, and even more preferably from 0.10 to 0.50% by mass.

More specifically, in a case in which n²⁰¹ in Formula (M) is 0, the ring structure between Sp²⁰¹ and Sp²⁰² is preferably Formula (XXa-1) to Formula (XXa-5), even more preferably from Formula (XXa-1) to Formula (XXa-3), and even more preferably Formula (XXa-1) or Formula (XXa-2). However, both terminals of Formulas are chemically bonded to Sp²⁰¹ or Sp²⁰².

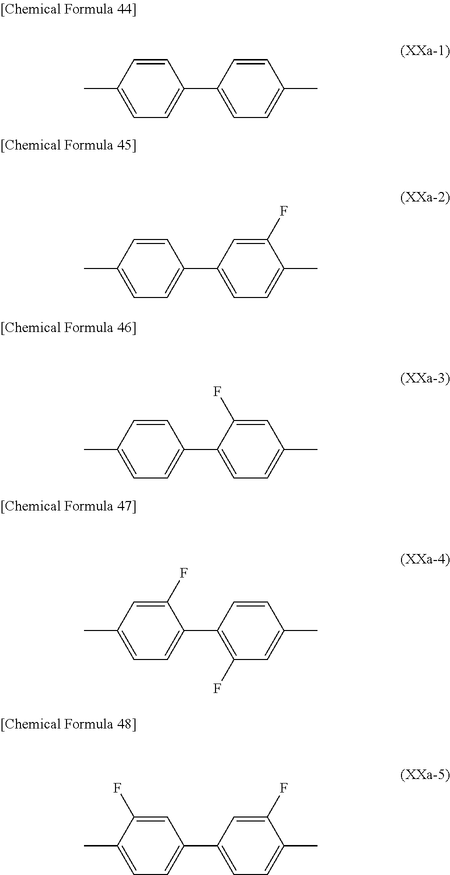

The alignment regulating force of the polymerizable compound represented by Formula (M) containing these backbones after the polymerization is optimal for a liquid crystal display device having a PSA mode, and there is an effect that the display unevenness is suppressed or does not occur at all since a favorable alignment state is obtained.

From the facts described above, as the polymerizable monomer, compounds represented by Formula (XX-1) to Formula (XX-10) are preferable and compounds represented by Formula (XX-1) to Formula (XX-4) are even more preferable.

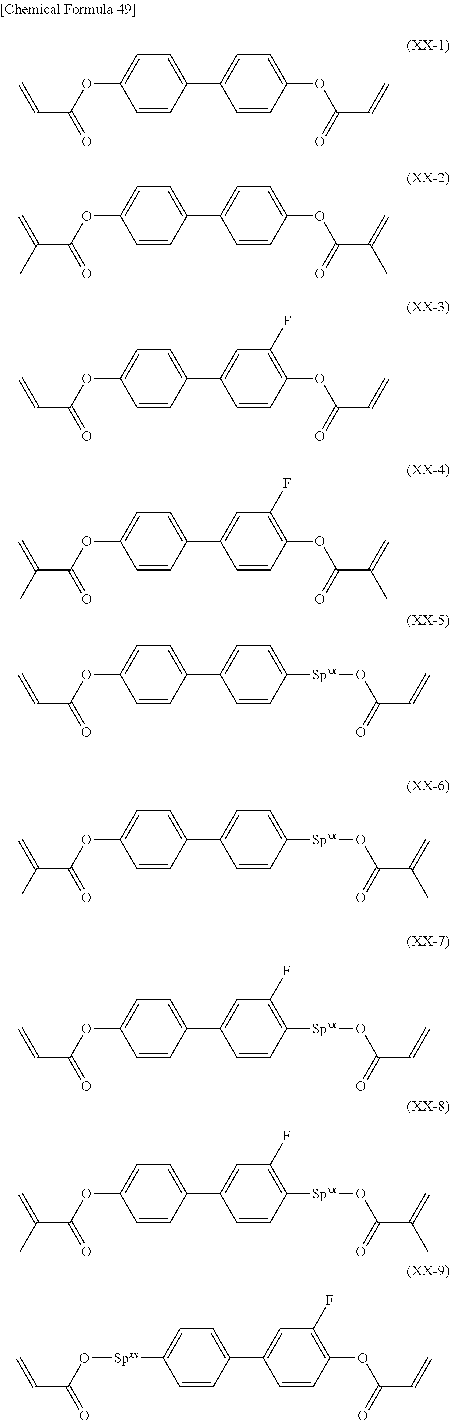

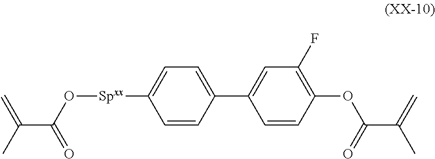

In Formulas, Sp^{xx} represents an alkylene group having from 1 to 8 carbon atoms or —O— (CH₂)ₛ— (in the formula, s represents an integer from 2 to 7 and the oxygen atom is chemically bonded to a ring).

The hydrogen atom in the phenyl group in Formulas may be further substituted with —F, —Cl, —CF₃, —CH₃, or any of Formula (R-1) to Formula (R-15).

In a case in which n²⁰¹ in Formula (M) is 1, for example, polymerizable compounds such as Formula (M31) to Formula (M48) are preferable.

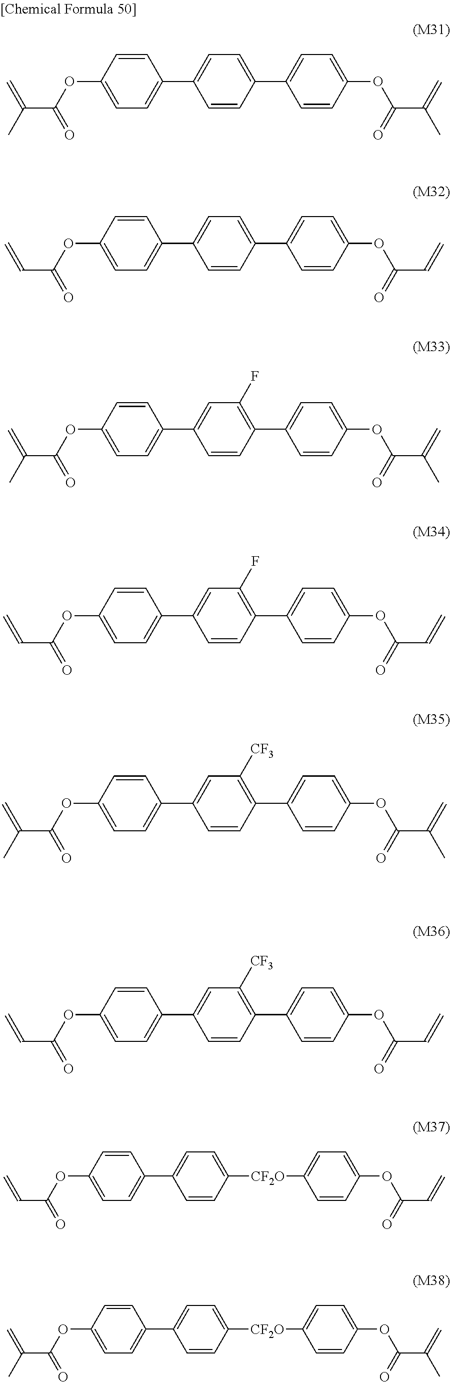

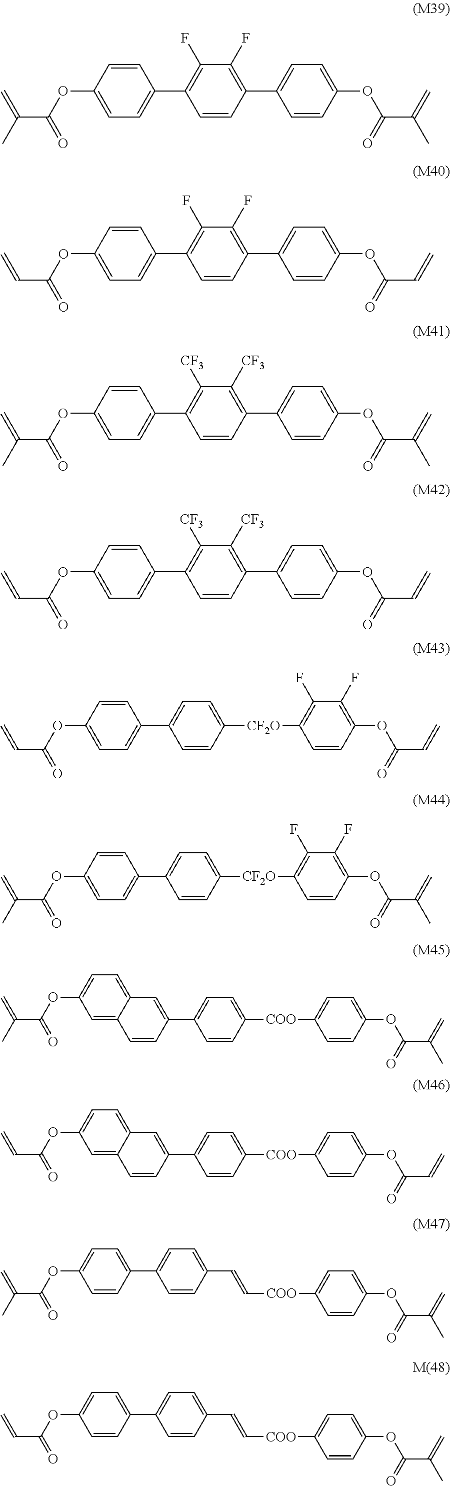

The hydrogen atom in the phenyl group and the naphthalene group in Formulas may be further substituted with —F, —Cl, —CF₃, —CH₃, or any of Formula (R-1) to Formula (R-15).

The alignment regulating force of the polymerizable compound represented by Formula (M) containing these backbones after the polymerization is optimal for a liquid crystal display device having a PSA mode, and there is an effect that the display unevenness is suppressed or does not occur at all since a favorable alignment state is obtained.

In a case in which n²⁰¹ in Formula (M) is 1 and Formula (M) has a plurality of Formulas (R-1) or Formulas (R-2), for example, polymerizable compounds such as Formula (M301) to Formula (M316) are preferable.

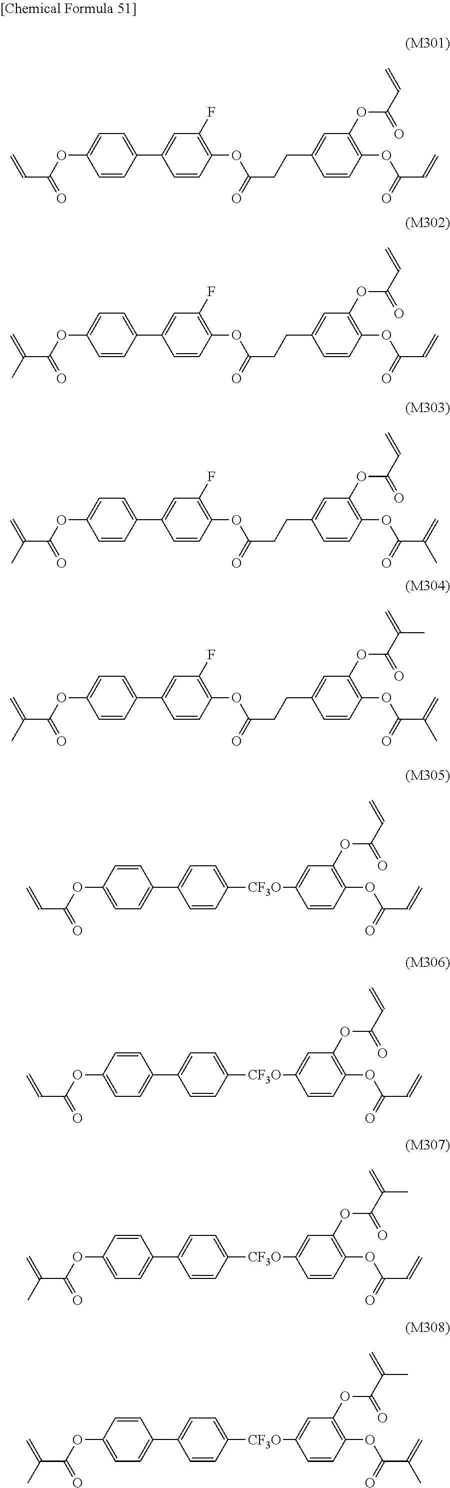

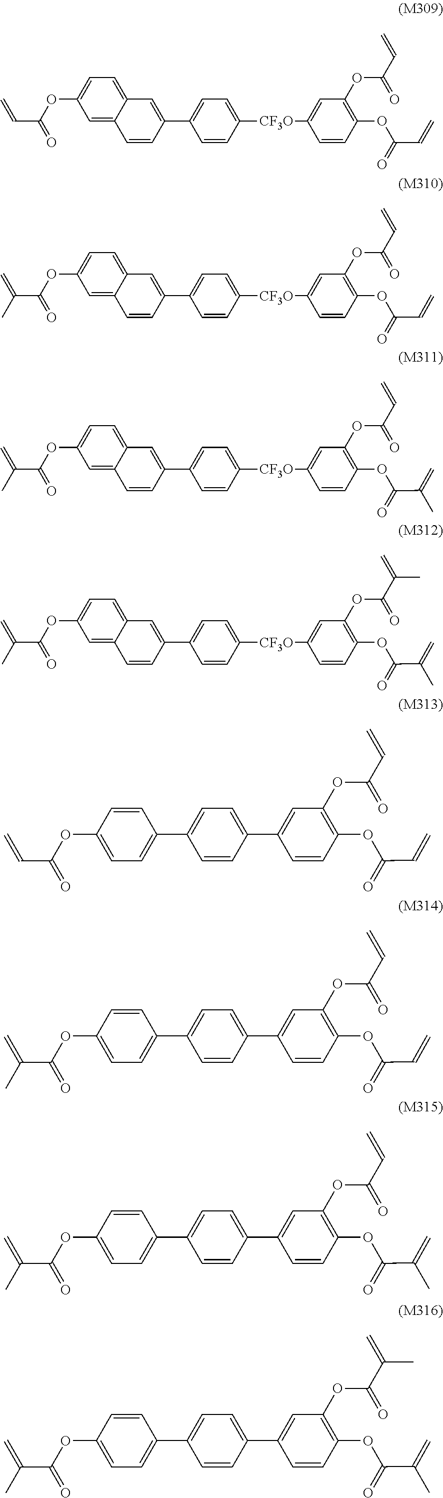

The hydrogen atom in the phenyl group and the naphthalene group in Formulas may be further substituted with —F, —Cl, —CF₃, or —CH₃

As the polymerizable compound represented by Formula (M) according to the present invention, for example, polymerizable compounds, such as Formula (Ia-1) to Formula (Ia-31) are also preferable.

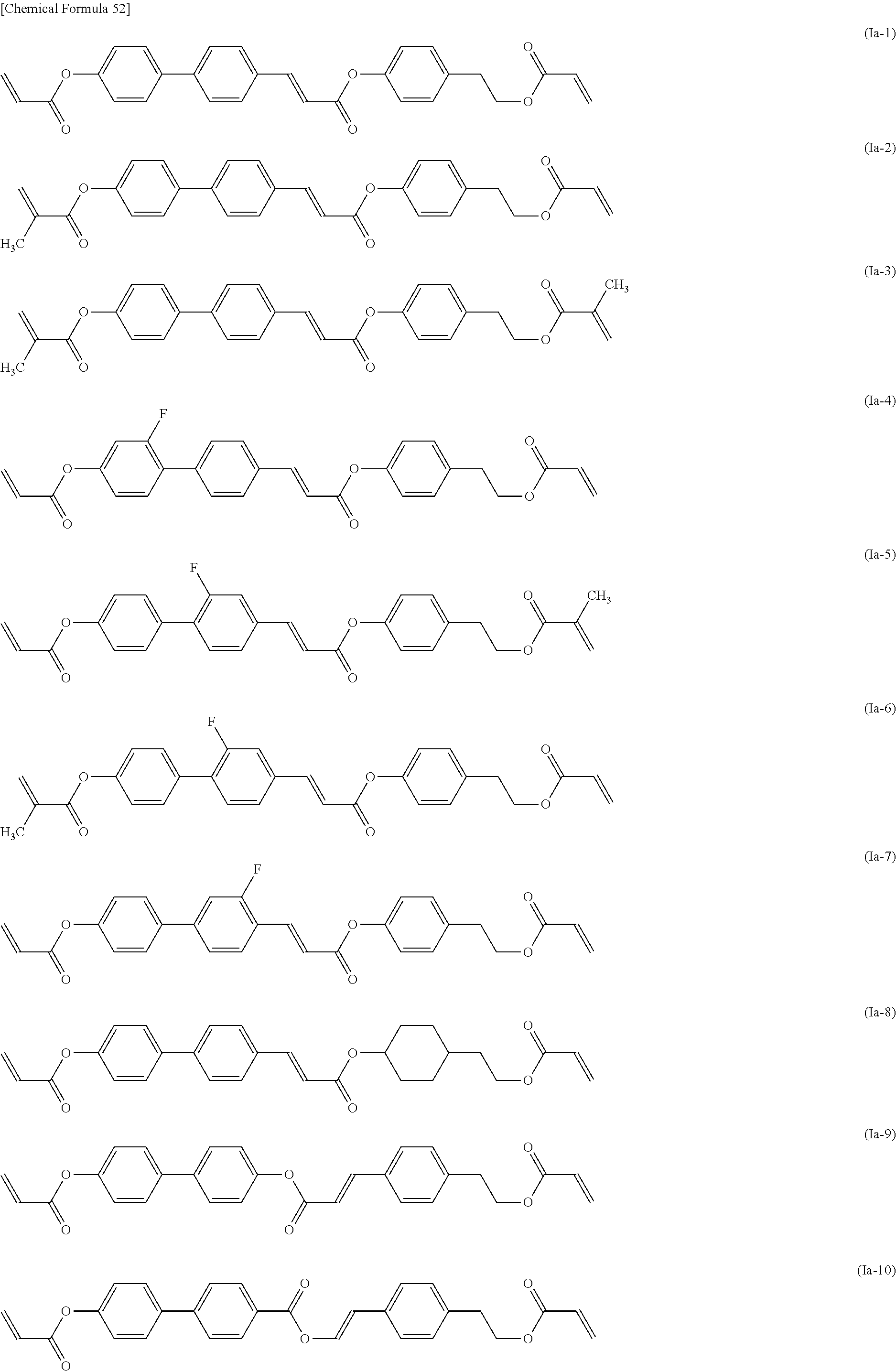

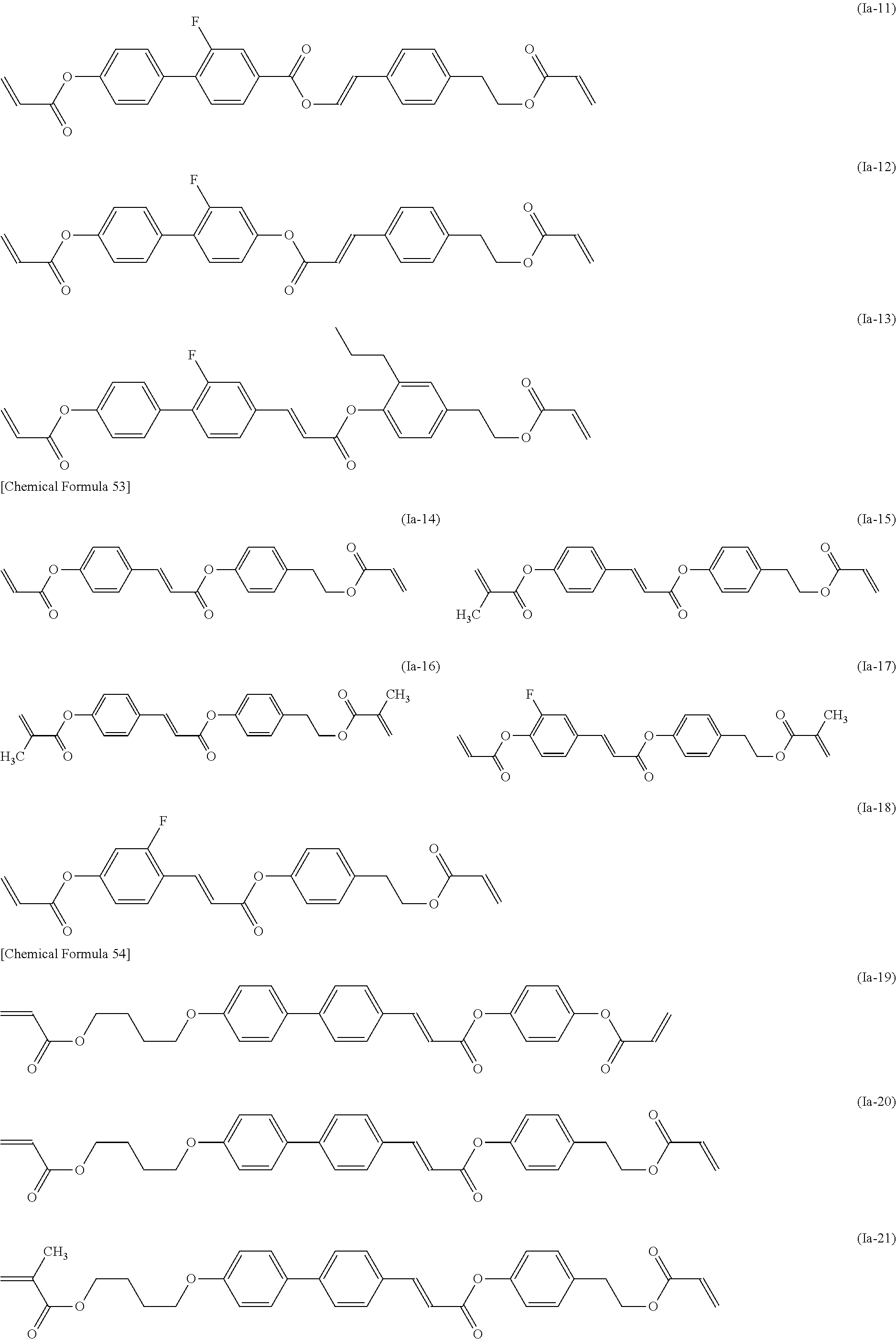

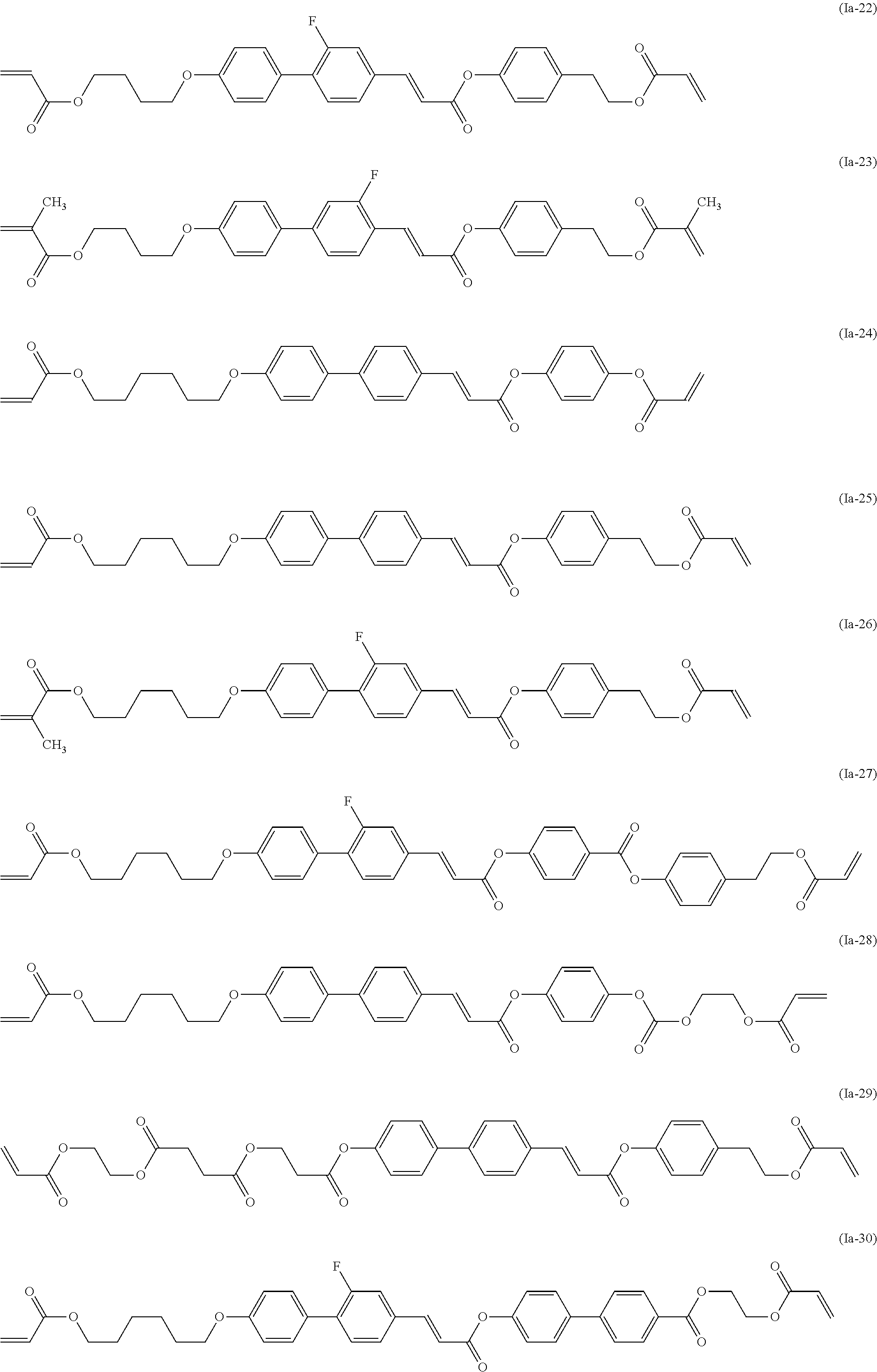

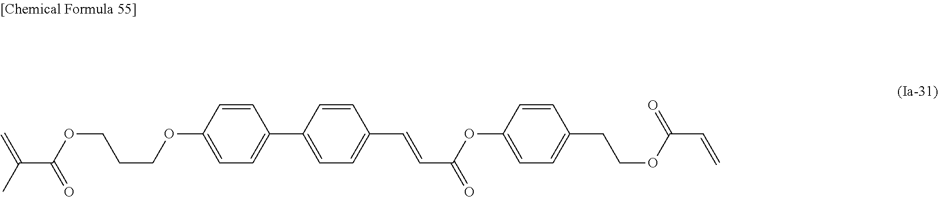 [SMILES: C=CC(=O)OCCc1ccc(OC(=O)/C=C/c2ccc(-c3ccc(OCCCOC(=O)C(=C)C)cc3)cc2)cc1]

The alignment regulating force of the polymerizable compound represented by Formula (M) containing these backbones after the polymerization is optimal for a liquid crystal display device having a PSA mode, and there is an effect that the display unevenness is suppressed or does not occur at all since a favorable alignment state is obtained.

In the liquid crystal composition according to the present invention, the polymerizable compound-containing liquid crystal composition containing Formula (M) that is the polymerizable compound can obtain a low viscosity (η), a low rotational viscosity (γ₁), and a great elastic constant (K₃₃), and thus a liquid crystal display device of a PSA mode or PSVA mode which uses this can realize high speed response.

A liquid crystal display device using the liquid crystal composition of the present invention has a remarkable feature of high speed response, in particular, it is useful as an active matrix driving liquid crystal display device, and it can be applied for a TN mode, a VA mode, a PSVA mode, a PSA mode, an IPS mode, a FFS mode, or an ECB mode.

In the case of adding a polymerizable compound to the liquid crystal composition of the present invention, the polymerization proceeds even in a case in which a polymerization initiator is not present but a polymerization initiator may be contained in order to promote the polymerization. Examples of the polymerization initiator may include a benzoin ether, a benzophenone, an acetophenone, a benzyl ketal, and an acylphosphine oxide.

Hereinafter, a liquid crystal display device of a horizontal electric field mode will be described with reference to the drawings as a preferred aspect of the liquid crystal display device according to the present invention, but the liquid crystal display device according to the present invention is not limited thereto.

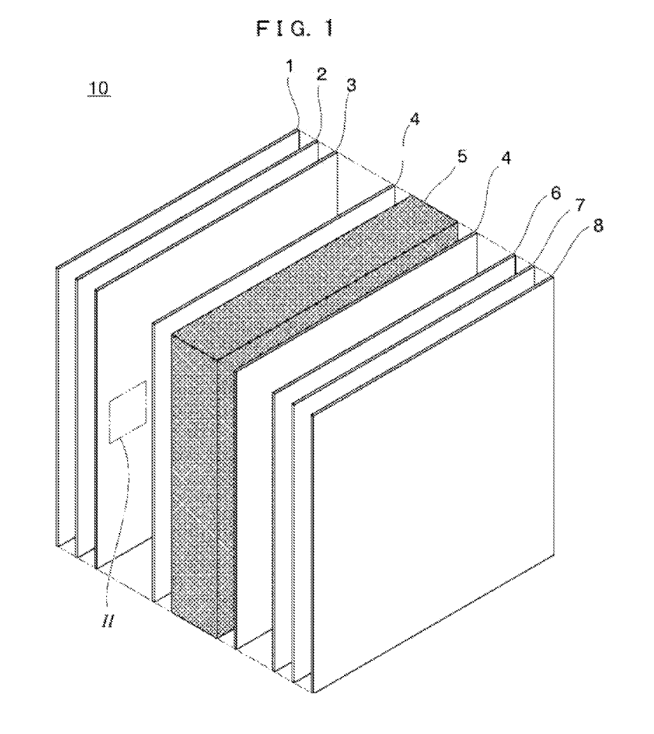

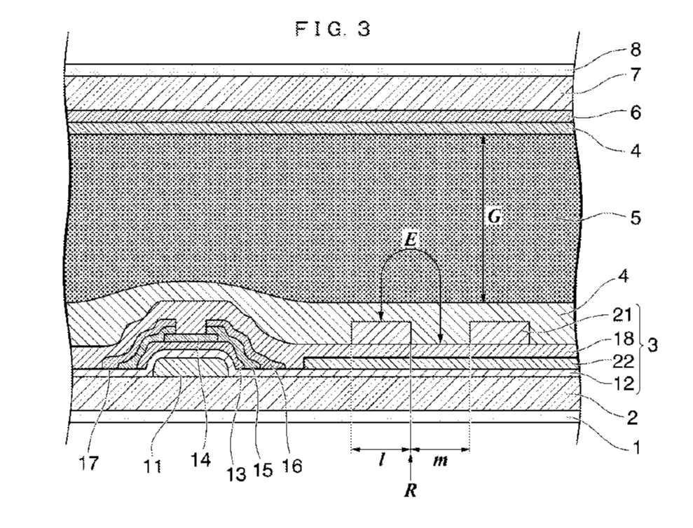

FIG. 1 is a view schematically illustrating the configuration of a liquid crystal display device. In FIG. 1, the respective constituent elements are described apart from one another for convenience of explanation. The configuration of a liquid crystal display device 10 according to the present invention is a liquid crystal display device of a horizontal electric field mode (FFS mode as an example in the drawing) having a liquid crystal composition (or a liquid crystal layer 5) sandwiched between a first transparent insulating substrate 2 and a second transparent insulating substrate 7 that are disposed to face each other as described in FIG. 1, and it is characterized by using the liquid crystal composition of the present invention as the liquid crystal composition. An electrode layer 3 is formed on the surface on the liquid crystal layer 5 side of the first transparent insulating substrate 2. In addition, a pair of alignment films 4 to induce homogeneous alignment by directly abutting on the liquid crystal composition constituting the liquid crystal layer 5 are disposed between the liquid crystal layer 5 and the first transparent insulating substrate 2 and between the liquid crystal layer 5 and the second transparent insulating substrate 8, and the liquid crystal molecule in the liquid crystal composition is aligned so as to be substantially parallel to the substrates 2 and 7 at the time of not applying a voltage. As illustrated in FIGS. 1 and 3, the second substrate 7 and the first substrate 2 may be sandwiched between a pair of polarizing plates 1 and 8. Furthermore, in FIG. 1, a color filter 6 is provided between the second substrate 7 and the alignment film 4. Incidentally, a form of the liquid crystal display device according to the present invention may be a so-called color filter on array (COA), or a color filter may be provided between the electrode layer including a thin film transistor and the liquid crystal layer or a color filter may be provided between the electrode layer including a thin film transistor and the second substrate.

In other words, the liquid crystal display device 10 according to the present invention has a configuration in which the first polarizing plate 1, the first substrate 2, the electrode layer 3 including a thin film transistor, the alignment film 4, the liquid crystal layer 5 containing a liquid crystal composition, the alignment film 4, the color filter 6, the second substrate 7, and the second polarizing plate 8 are sequentially stacked.

It is possible to use a transparent material exhibiting flexibility such as glass or plastic as the first substrate 2 and the second substrate 7, and either of them may be an opaque material such as silicone. Two substrates 2 and 7 are bonded to each other with a sealing material and an encapsulating material such as an epoxy-based thermosetting composition disposed in the peripheral region, and, for example, a particulate spacer such as glass particles, plastic particles, alumina particles or a spacer support composed of a resin formed by photolithography may be disposed between the substrates in order to keep the distance therebetween.

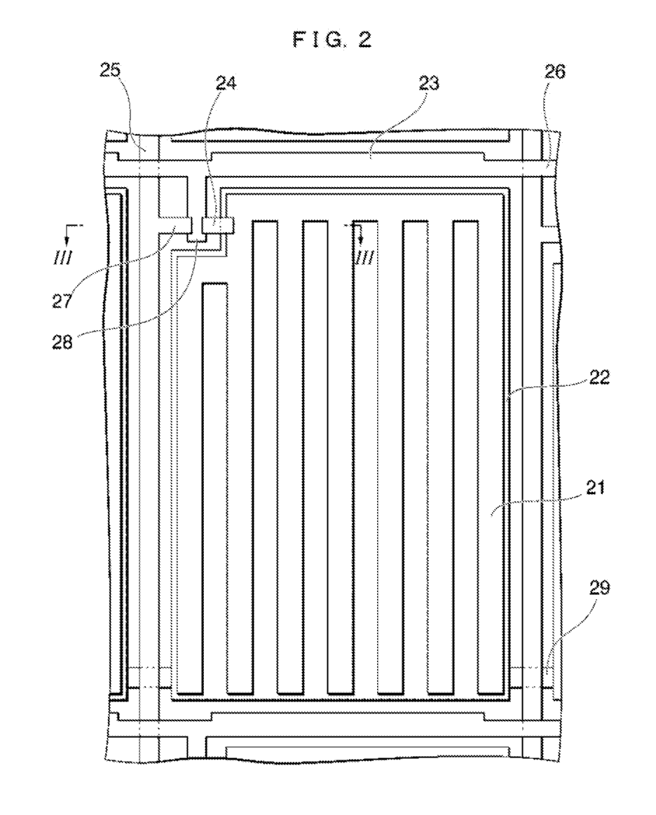

FIG. 2 is an enlarged plane view of a region surrounded by line II in the electrode layer 3 formed on the substrate 2 in FIG. 1. FIG. 3 is a cross-sectional view of the liquid crystal display device that is illustrated in FIG. 1 and cut in the line III-III direction in FIG. 2. As illustrated in FIG. 2, a plurality of gate bus lines 26 for supplying a scanning signal and a plurality of data bus lines 25 for supplying a display signal are disposed in the electrode layers 3 that is formed on the surface of the first substrate 2 and includes a thin film transistor in a matrix shape to intersect each other. Incidentally, in FIG. 2, only a pair of gate bus lines 25 and a pair of data bus lines 24 are illustrated.

The unit pixel of the liquid crystal display device is formed by the region surrounded by a plurality of gate bus lines 26 and a plurality of data bus lines 25, and a pixel electrode 21 and a common electrode 22 are formed in the unit pixel. A thin film transistor including a source electrode 27, a drain electrode 24, and a gate electrode 28 is provided in the vicinity of intersecting portion at which the gate bus lines 26 and the data bus lines 25 intersect each other. This thin film transistor is linked to the pixel electrode 21 as a switching element for supplying a display signal to the pixel electrode 21. In addition, a common line 29 is provided in parallel to the gate bus lines 26. This common line 29 is linked to the common electrode 22 for supplying a common signal to the common electrode 22.

A preferred aspect of the structure of the thin film transistor, for example, as illustrated in FIG. 3, includes a gate electrode 11 formed on the surface of the substrate 2, a gate insulating layer 12 provided so as to cover the gate electrode 11 and to cover substantially the entire surface of the substrate 2, a semiconductor layer 13 formed on the surface of the gate insulating layer 12 so as to face the gate electrode 11, a protective film 14 provided so as to cover a part of the surface of a semiconductor layer 17, a drain electrode 16 provided so as to cover the side end portion of either of the protective layer 14 or the semiconductor layer 13 and to be in contact with the gate insulating layer 12 formed on the surface of the substrate 2, a source electrode 17 provided so as to cover the side end portion of the other of the protective film 14 or the semiconductor layer 13 and to be in contact with the gate insulating layer 12 formed on the surface of the substrate 2, and an insulating protective layer 18 provided so as to cover the drain electrode 16 and the source electrode 17. An anodic oxide coating (not illustrated) may be formed on the surface of the gate electrode 11 for the reason to eliminate a step with the gate electrode, and the like.

Amorphous silicon, polycrystalline polysilicon, and the like can be used in the semiconductor layer 13, but it is preferable to use a transparent semiconductor film such as ZnO, IGZO (In—Ga—Zn—O), or ITO from the viewpoint of being able to suppress the adverse effect of the optical carrier caused by light absorption and increasing the aperture ratio of the device as well.

Furthermore, an ohmic contact layer 15 may be provided between the semiconductor layer 13 and the drain electrode 16 or the source electrode 17 in order to decrease the width or height of the Schottky barrier. It is possible to use a material obtained by adding an impurity such as phosphorus to n-type amorphous silicon, n-type polycrystalline polysilicon, or the like at a high concentration in the ohmic contact layer.

The gate bus lines 26 or the data bus lines 25 and the common line 29 are preferably a metal film and more preferably Al, Cu, Au, Ag, Cr, Ta, Ti, Mo, W, Ni, or an alloy thereof, and it is even more preferable to use a wire of Al or an alloy thereof. In addition, the insulating protective layer 18 is a layer having an insulating function, and it is formed of silicon nitride, silicon dioxide, a silicon oxynitride film, or the like.

In the embodiment illustrated in FIGS. 2 and 3, the common electrode 22 is a plate-shaped electrode formed on substantially the entire surface of the gate insulating layer 12, meanwhile, the pixel electrode 21 is a comb-shaped electrode formed on the insulating protective layer 18 which covers the common electrode 22. In other words, the common electrode 22 is disposed at the position closer to the first substrate 2 than the pixel electrode 21, and these electrodes are disposed to overlap each other via the insulating protective layer 18. The pixel electrode 21 and the common electrode 22 are, for example, formed of a transparent conductive material such as ITO (Indium Tin Oxide), IZO (Indium Zinc Oxide), or IZTO (Indium Zinc Tin Oxide). The area to be opened in the unit pixel area increases and the aperture ratio and the transmittance increase since the common electrode 22 and the pixel electrode 21 are formed of a transparent conductive material.

In addition, the pixel electrode 21 and the common electrode 22 are formed such that the distance between electrodes (also referred to as the minimum separation distance): R between the pixel electrode 21 and the common electrode 22 is smaller than the distance between the first substrate 2 and the second substrate 7: G in order to form a fringe electric field between the electrodes. Here, the distance between electrodes: R refers to the distance between the respective electrodes in the horizontal direction to the substrate. In FIG. 3, the plate-shaped common electrode 22 and the comb-shaped pixel electrode 21 overlap each other and thus an example in which the distance between electrodes: R=0 is illustrated, and the minimum separation distance: R is smaller than the distance between the first substrate 2 and the second substrate 7 (namely, cell gap): G and thus the fringe electric field E is formed. Accordingly, a FFS type liquid crystal display device can utilize the horizontal electric field formed in a vertical direction to the line for forming the comb shape of the pixel electrode 21 and a parabolic electric field. It is preferable to form the electrode width: l of the comb-shaped portion of the pixel electrode 21 and the gap width: m of the comb-shaped portion of the pixel electrode 21 into a width to an extent to which the entire liquid crystal molecules in the liquid crystal layer 5 can be driven by the electric field to be generated. In addition, the minimum separation distance R between the pixel electrode and the common electrode can be adjusted as the (average) thickness of the gate insulating film 12. In addition, the liquid crystal display device according to the present invention, unlike FIG. 3, may be formed (IPS mode) such that the distance between electrodes (also referred to as the minimum separation distance): R between the pixel electrode 21 and the common electrode 22 is greater than the distance between the first substrate 2 and the second substrate 7: G. In this case, for example, a configuration in which the comb-shaped pixel electrode and the comb-shaped common electrode are provided so as to be alternate in substantially the same plane, and the like are mentioned.

The liquid crystal display device according to the present invention preferably has the liquid crystal display composition of a FFS mode utilizing a fringe electric field, and a fringe electric field is formed between the common electrode and the pixel electrode when the shortest separation distance d between the common electrode 22 and the pixel electrode 21 that are adjacent to each other is shorter than the shortest separation distance D between the alignment layers 4 (distance between substrates), and thus it is possible to efficiently utilize the alignment of the liquid crystal molecules in the horizontal and vertical directions. In the case of the liquid crystal display device of a FFS mode as a preferred form of the present invention, the equipotential line of the parabolic electric field between the pixel electrode 21 and the common electrode 22 is formed to the upper portion of the pixel electrode 21 and the common electrode 22 and the major axis of the liquid crystal molecules in the liquid crystal layer 5 is arranged along the electric field formed when a voltage is applied to the liquid crystal molecules disposed such that the major axis direction is parallel to the alignment direction of the alignment layer. In particular, the liquid crystal composition according to the present invention uses a liquid crystal molecule which exhibits a positive dielectric anisotropy, and thus the major axis direction of the liquid crystal molecules is arranged along the electric field direction generated.

It is preferable to form a black matrix (not illustrated) at the part coping with the thin film transistor and a storage capacitor 23 of the color filter 6 from the viewpoint of preventing the leakage of light.

A pair of alignment films 4 to induce homogeneous alignment by directly abutting on the liquid crystal composition constituting the liquid crystal layer 5 is provided on the electrode layer 3 and the color filter 6.

In addition, the polarizing plate 1 and the polarizing plate 8 can be adjusted so as to have a favorable viewing angle or contrast by adjusting the polarization axis of the respective polarizing plates, and they preferably have transmission axes orthogonal to each other such that the transmission axes thereof are operated in a normally black mode. In particular, it is preferable that either of the polarizing plate or the polarizing plate 8 is disposed so as to have a transmission axis parallel to the alignment direction of the liquid crystal molecule 30. In addition, it is preferable to adjust the product of the refractive index anisotropy Δn and the cell thickness d of the liquid crystal so as to maximize the contrast. Furthermore, it is also preferable to use a phase difference film for extending the viewing angle.

### EXAMPLES

Hereinafter, the present invention will be described in more detail with reference to Examples, but the present invention is not limited to these Examples. In addition, the term “%” in the compositions of the following Examples and Comparative Examples means “% by mass”

The properties measured in Examples are as follows.

Tₙᵢ: nematic phase-isotropic liquid phase transition temperature (° C.)

T_{cn}: solid phase-nematic phase transition temperature (° C.)

Δn: refractive index anisotropy at 20° C.

Δ∈: dielectric anisotropy at 20° C.

η: viscosity at 20° C. (mPa·s)

γ1: rotational viscosity at 20° C. (mPa·s)

VHR (Init): voltage holding ratio (%) at 60° C. under condition having frequency of 60 Hz and applied voltage of 1 V

VHR (HEAT): voltage holding ratio (%) at 60° C. under condition having frequency of 60 Hz and applied voltage of 1 V measured after being left to stand for 48 hours in a state of being at 100° C.

Drop mark: as the evaluation on drop mark of the liquid crystal display device, the drop mark standing out white in the case of displaying whole black was visually evaluated according to the following four stages.

⊙: There is no drop mark.

◯: There are a little bit drop marks in a level to be acceptable.

Δ: There are drop marks in a level to be unacceptable.

x: There are drop marks in a fairly poor level.

Linear residual image: as the linear residual image test of the liquid crystal displaydevice, the level of linear residual image generated at the boundary portion of the fixed pattern when a predetermined fixed pattern was displayed in a display area for 2000 hours was visually evaluated according to the following four stages.

⊙: There is no linear residual image.

∘: There are a little bit linear residual images in a level to be acceptable.

Δ: There are linear residual images in a level to be unacceptable.

x: There are linear residual images in a fairly poor level. (Side chain)

—F —F Fluorine atom

F— —F Fluorine atom

-n —CₙH₂ₙ₊₁ linear alkyl group having n carbon atoms

n- —CₙH₂ₙ₊₁— linear alkyl group having n carbon atoms

—On —OCₙH₂ₙ₊₁ linear alkoxy group having n carbon atoms

nO— CₙH₂ₙ₊₁O— linear alkoxy group having n carbon atoms

—V —CH═CH₂

V— CH₂═CH—

—V1 —CH═CH—CH₃

1V— CH₃—CH═CH—

-2V —CH₂—CH₂—CH═CH₃

V2— CH₃═CH—CH₂—CH₂—

-2V1 —CH₂—CH₂—CH═CH—CH₃

1V2— CH₃—CH═CH—CH₂—CH₂

(Linking Group)

—CF₂O— —CF₂—O—

—OCF2- —O—CF₂—

-1O— —CH₂—O—

—O1- —O—CH₂—

(Ring Structure)

[Chemical Formula 56]

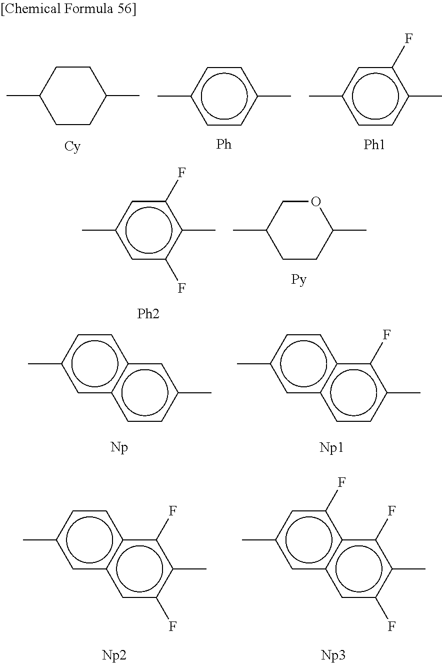

### Example 1, Comparative Example 1, and Comparative Example 2

Liquid crystal compositions LC-1 (Example 1), LC-A (Comparative Example 1), and LC-B (Comparative Example 2) were prepared. The measurement of the physical property values, measurement of VHR (Init) and VHR (HEAT), and evaluation on the presence or absence of drop marks and the presence or absence of linear residual images of these were conducted, and the results thereof were as follows.

TABLE 1

| | | | Comparative | Comparative |
|---|---|---|---|---|
| | | Example 1 | Example 1 | Example 2 |
| | | LC-1 | LC-A | LC-B |
| Antioxidant | Formula (II-2) | 0.02 | — | — |
| Antioxidant | Formula (St-1) | — | 0.02 | — |
| Antioxidant | Formula St-2) | — | — | 0.02 |
| 3-Ph—Ph1—Ph2—CF2O—Np2—F | Formula (N3-1) | 3 | 3 | 3 |
| 4-Ph—Ph1—Ph2—CF2O—Np2—F | Formula (N3-1) | 3 | 3 | 3 |
| 2-Ph—Ph2—CF2O—Np2—F | Formula (N3-3) | 2 | 2 | 2 |
| 3-Ph—Ph2—CF2O—Np2—F | Formula (N3-3) | 2 | 2 | 2 |
| 3-Cy-Cy-V | Formula (III-A) | 36.98 | 36.98 | 36.98 |
| 3-Cy-Ph—O1 | Formula (III-D) | 4 | 4 | 4 |
| 3-Ph—Ph-1 | Formula (III-F) | 6 | 6 | 6 |
| 5-Ph—Ph-1 | Formula (III-F) | 11 | 11 | 11 |
| V-Cy-Cy-Ph-1 | Formula (III-G) | 3 | 3 | 3 |
| 3-Cy-Cy-Ph-1 | Formula (III-G) | 6 | 6 | 6 |
| 3-Cy-Ph—Ph-2 | Formula (III-H) | 3 | 3 | 3 |
| V-Cy-Ph—Ph-3 | Formula (III-H) | 11 | 11 | 11 |
| IV-Cy-Ph—Ph-3 | Formula (III-H) | 5 | 5 | 5 |
| 2-Cy-Cy-Ph—Ph1—F | Formula (Pa) | 4 | 4 | 4 |

| | | | |
|---|---|---|---|
| Sum | 100 | 100 | 100 |
| Tni [° C.] | 75.8 | 75.7 | 75.7 |
| Tcn [° C.] | −21 | −21 | −21 |
| Δn | 0.117 | 0.117 | 0.117 |
| η[mPa · s] | 9.9 | 9.9 | 9.9 |
| γ1[mPa · s] | 40 | 40 | 40 |
| Δε | 2.4 | 2.4 | 2.4 |
| VHR (Init) | 99.9 | 99.8 | 99.8 |
| VHR (HEAT) | 99.9 | 99.5 | 99.7 |
| Drop mark | ⊙ | Δ | ◯ |
| Linear residual image | ⊙ | Δ | Δ |

Incidentally, the antioxidants (St-1) and (St-2) are compounds represented by the following formulas.

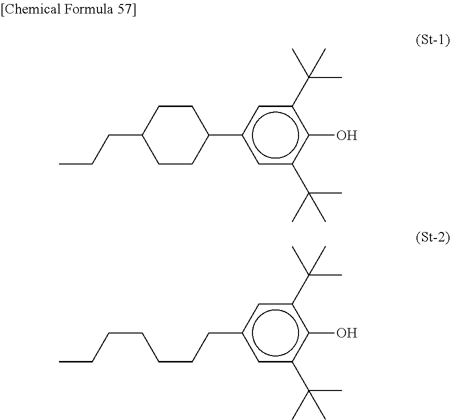

A significant difference has been confirmed in VHR (HEAT), the presence or absence of drop marks, and the presence or absence of linear residual images. From these results, it has been confirmed that the liquid crystal composition LC-1 of the present invention is a liquid crystal composition which has a liquid crystal phase in a wide temperature range, exhibits low viscosity and favorable solubility at a low temperature, has a high specific resistance or a high voltage holding ratio and a positive Δ∈, and is stable with respect to heat or light, and a liquid crystal display device using this exhibits excellent display quality and hardly causes display defects such as a drop mark or a linear residual image.

### Example 2, Example 3, Example 4, Example 5, Example 6, and Comparative Example 3

Liquid crystal compositions LC-2 (Example 2), LC-3 (Example 3), LC-4 (Example 4), LC-5 (Example 5), LC-6 (Example), and LC-C(Comparative Example 3) were prepared. The measurement of the physical property values, measurement of VHR (Init) and VHR (HEAT), and evaluation on the presence or absence of drop marks and the presence or absence of linear residual images of these were conducted, and the results thereof were as follows.

TABLE 2

| | | Example | Example | Example | Example | Comparative | Example |
|---|---|---|---|---|---|---|---|
| | | 2 | 3 | 4 | 5 | Example 3 | 6 |
| | | LC-2 | LC-3 | LC-4 | LC-5 | LC-C | LC-6 |
| Antioxidant | Formula (II-2) | 0.01 | 0.05 | 0.01 | 0.02 | — | 0.12 |
| 3-Ph—Ph1—Ph2—CF2O—Np2—F | Formula (N3-1) | 3 | 3 | — | 4 | 4 | — |
| 4-Ph—Ph1—Ph2—CF2O—Np2—F | Formula (N3-1) | 3 | 3 | — | 4 | 4 | — |
| 2-Ph—Ph2—CF2O—Np2—F | Formula (N3-3) | 3 | 3 | — | 5 | 5 | — |
| 3-Ph—Ph2—CF2O—Np2—F | Formula (N3-3) | 8 | 8 | 10 | 10 | 10 | 10 |
| 4-Ph—Ph2—CF2O—Np2—F | Formula (N3-3) | 3 | 3 | — | 5 | 5 | — |
| 3-Cy-Cy-2 | Formula (III-A) | — | 3 | — | — | — | — |
| 3-Cy-Cy-4 | Formula (III-A) | — | 3 | — | — | — | — |
| 3-Cy-Cy-V | Formula (III-A) | 43.99 | 43.95 | 37.99 | 37.98 | 38 | 43.88 |
| 2-Cy-Cy-V1 | Formula (III-A) | — | — | 3 | — | — | — |
| 3-Cy-Cy-V1 | Formula (III-A) | — | — | 3 | — | — | — |
| 3-Ph—Ph-1 | Formula (III-F) | — | — | 3 | — | — | 6 |
| 5-Ph—Ph-1 | Formula (III-F) | — | 5 | 3 | — | — | — |
| V-Cy-Cy-Ph-1 | Formula (III-G) | — | 2.5 | — | — | — | — |
| 3-Cy-Cy-Ph-1 | Formula (III-G) | 5 | 2.5 | 7 | 7 | 7 | 4 |
| 3-Cy-Ph—Ph-2 | Formula (III-H) | — | — | 4 | — | — | 6 |
| V-Cy-Ph—Ph-3 | Formula (III-H) | 5 | 2 | 8 | 8 | 8 | 6 |
| 2-PH2—O1-Cy-Ph2—Ph2—F | Formula (K11) | — | — | 3 | — | — | 3 |
| 3-Ph2—O1-Cy-Ph2—Ph2—F | Formula (K11) | — | — | 3 | — | — | 3 |
| 2-Ph2—O1—Ph—Np2—F | Formula (K25) | — | — | — | 3 | 3 | — |
| 3-Ph2—O1—Ph—Np2—F | Formula (K25) | — | — | — | 4 | 4 | — |
| 2-Cy-Cy-Ph1—F | Formula (P25) | 8 | — | — | — | — | — |
| 3-Cy-Cy-Ph1—F | Formula (P25) | 18 | 16 | 10 | — | — | 15 |
| 5-Cy-Cy-Ph1—F | Formula (P25) | — | — | 5 | — | — | — |
| 2-Cy-Cy-Ph—Ph1—F | Formula (Pa) | — | — | — | 4 | 4 | — |
| 3-Cy-Cy-Ph—Ph1—F | Formula (Pa) | — | 2 | — | 4 | 4 | — |
| 4-Cy-Ph—Ph—Ph1—F | Formula (Pa) | — | — | — | 4 | 4 | — |
| 3-Cy-Ph—Ph1-Cy-2 | Formula (VII-c) | — | — | — | — | — | 1.5 |
| 3-Cy-Cy-COO—Ph-Cy-3 | Formula (V-9.1) | — | — | — | — | — | 1.5 |

| | | | | | | |
|---|---|---|---|---|---|---|
| Sum | 100 | 100 | 100 | 100 | 100 | 100 |
| Tni [° C.] | 82.9 | 76.5 | 77.7 | 109.8 | 109.8 | 78.7 |
| Tcn [° C.] | −26 | −26 | −31 | −33 | −33 | −31 |
| Δn | 0.096 | 0.095 | 0.100 | 0.140 | 0.140 | 0.100 |
| η [mPa · s] | 11.9 | 10.5 | 10.4 | 18.9 | 18.9 | 10.5 |
| γ1 [mPa · s] | 85 | 79 | 80 | 158 | 158 | 77 |
| Δε | 6.2 | 5.3 | 4.8 | 9.8 | 9.8 | 4.6 |
| VHR (Init) | 99.7 | 99.7 | 99.8 | 99.6 | 99.6 | 99.6 |
| VHR (HEAT) | 99.5 | 99.4 | 99.4 | 99.3 | 98.1 | 99.5 |
| Drop mark | ◯ | ⊙ | ◯ | ◯ | Δ | ◯ |
| Linear residual image | ◯ | ⊙ | ⊙ | ◯ | X | ◯ |

A significant difference has been confirmed in VHR (HEAT), the presence or absence of drop marks, and the presence or absence of linear residual images. From these results, it has been confirmed that the liquid crystal compositions LC-2, LC-3, LC-4, and LC-5 of the present invention are liquid crystal compositions which have a liquid crystal phase in a wide temperature range, exhibit low viscosity and favorable solubility at a low temperature, have a high specific resistance or a high voltage holding ratio and a positive Δ∈, and are stable with respect to heat or light, and liquid crystal display devices using these exhibit excellent display quality and hardly cause display defects such as a drop mark or a linear residual image.

### REFERENCE SIGNS LIST

1, 8: Polarizing plate
2: First substrate
3: Electrode layer
4: Alignment film
5: Liquid crystal layer
6: Color filter
6G: Green filter
6R: Red filter
7: Second substrate
11: Gate electrode
12: Gate insulating film
13: Semiconductor layer
14: Insulating layer
15: Ohmic contact layer
16: Drain electrode
17: Source electrode
18: Insulating protective layer
21: Pixel electrode
22: Common electrode
23: Storage capacitor
24: Drain electrode
25: Data bus line
27: Source bus line
29: Common line
30: Buffer layer

1. An antioxidant for liquid crystal composition comprising one or more kinds of liquid crystal compounds having a positive dielectric anisotropy of 4 or more as an absolute value at 3% or more, wherein
the antioxidant for liquid crystal composition is used in a liquid crystal composition, and
the antioxidant for liquid crystal composition is represented by Formula (II-1): 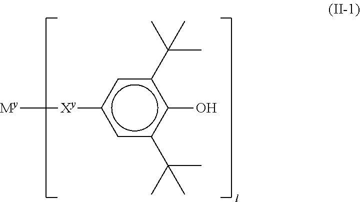
(in Formula (II-1), M^{y} represents a hydrocarbon having from 1 to 25 carbon atoms (one or two or more —CH₂— in the hydrocarbon may be substituted with —O—, —CO—, —COO—, or —OCO— so as not to be directly adjacent to an oxygen atom), a 1,4-phenylene group, or a trans-1,4-cyclohexylene group, wherein an arbitrary hydrogen atom in the 1,4-phenylene group may be substituted with a fluorine atom,
X^{y}'s may be the same as or different from one another and represent an alkylene group having from 1 to 15 carbon atoms (one or two or more —CH₂— in the alkylene group may be substituted with —O—, —CO—, —COO—, or —OCO— so as not to be directly adjacent to an oxygen atom), —OCH₂—, —CH₂O—, —COO—, —OCO—, —CF₂O—, —OCF₂—, —CF₂CF₂—, —CH═CH—COO—, —CH═CH—OCO—, —COO—CH═CH—, —OCO—CH═CH—, —CH═CH—, —C≡C—, a single bond, a 1,4-phenylene group, or a trans-1,4-cyclohexylene group, wherein an arbitrary hydrogen atom in the 1,4-phenylene group may be substituted with a fluorine atom, and
l represents an integer from 2 to 6).

2. The antioxidant for liquid crystal composition according to claim 1, wherein the Formula (II-1) is a compound represented by Formula (II-2): 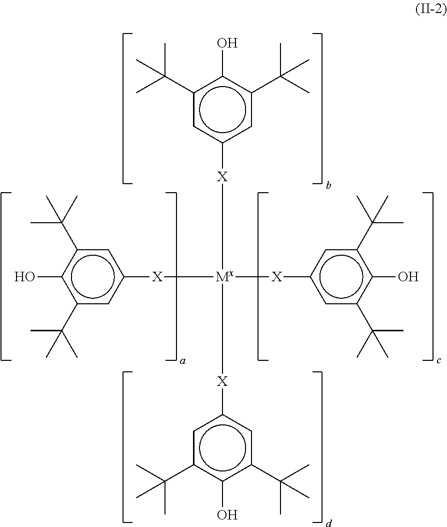
(in Formula (II-2), M^{x} represents a hydrocarbon having from 1 to 25 carbon atoms (one or two or more —CH₂— in the hydrocarbon may be substituted with —O—, —CO—, —COO—, or —OCO— so as not to be directly adjacent to an oxygen atom),
X's may be the same as or different from one another and represent an alkylene group having from 1 to 15 carbon atoms (one or two or more —CH₂— in the alkylene group may be substituted with —O—, —CO—, —COO—, or —OCO— so as not to be directly adjacent to an oxygen atom), —OCH₂—, —CH₂O—, —COO—, —OCO—, —CF₂O—, —OCF₂—, —CF₂CF₂—, —CH═CH—COO—, —CH═CH—OCO—, —COO—CH═CH—, —OCO—CH═CH—, —CH═CH—, —C≡C—, a single bond, a 1,4-phenylene group, or a trans-1,4-cyclohexylene group, wherein an arbitrary hydrogen atom in the 1,4-phenylene group may be substituted with a fluorine atom), and
a, b, c, and d each independently represent 0 or 1, but a+b+c+d represents 2 or more).

3. The antioxidant for liquid crystal composition according to claim 2, wherein a+b+c+d in the Formula (II-2) is from 2 to 4.

4. The antioxidant for liquid crystal composition according to claim 1, wherein the Formula (II-1) or the Formula (II-2) is a compound represented by Formula (II): 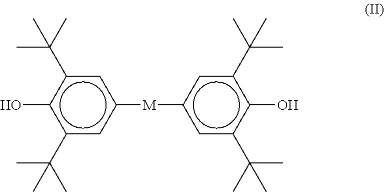
(in Formula (II), M represents an alkylene group having from 1 to 15 carbon atoms (one or two or more —CH₂— in the alkylene group may be substituted with —O—, —CO—, —COO—, or —OCO— so as not to be directly adjacent to an oxygen atom), —OCH₂—, —CH₂O—, —COO—, —OCO—, —CF₂O—, —OCF₂, —CF₂CF₂—, —CH═CH—COO—, —CH═CH—OCO—, —COO—CH═CH—, —OCO—CH═CH—, —CH═CH—, —C≡C—, a single bond, a 1,4-phenylene group, or a trans-1,4-cyclohexylene group, wherein an arbitrary hydrogen atom in the 1,4-phenylene group may be substituted with a fluorine atom).

5. The antioxidant for liquid crystal composition according to claim 1, wherein the compound represented by Formula (II-1) or Formula (II-2) is a compound represented by Formula (II-a):

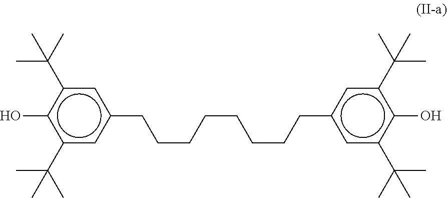

6. A liquid crystal composition comprising:
one or more kinds of compounds represented by Formula (N1): 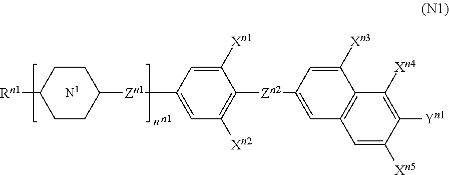
(In Formula (N1), Rⁿ¹ represents an alkyl group having from 1 to 10 carbon atoms or an alkenyl group having from 2 to 10 carbon atoms, wherein one or non-adjacent two or more —CH₂— in the groups may be each independently substituted with —C≡C—, —O—, —CO—, —COO—, or —OCO— and one or non-adjacent two or more hydrogen atoms in the groups may be each independently substituted with a fluorine atom,
Ring N¹ represents a 1,4-cyclohexylene group (one or non-adjacent two or more —CH₂— in the group may be substituted with —O— or —S—) or a 1,4-phenylene group (one or non-adjacent two or more —CH═ in the group may be substituted with —N═), wherein hydrogen atoms in the groups may be each independently substituted with a cyano group or a fluorine atom,
Zⁿ¹ and Zⁿ² each independently represent a single bond, —CH₂CH₂—, —(CH₂)₄—, —OCH₂—, —CH₂O—, —OCF₂—, —CF₂O—, —CH₂CH₂CF₂O—, —COO—, —OCO—, or —C≡C—,
nⁿ¹'s each independently represent an integer from 0 to 4, and Rings N¹ may be the same as or different from one another when nⁿ¹ is 2 or more,
Xⁿ¹, Xⁿ², Xⁿ³, Xⁿ⁴, and Xⁿ⁵ each independently represent a hydrogen atom or a fluorine atom, and
Yⁿ¹'s each independently represent a fluorine atom, a cyano group, a trifluoromethyl group, a fluoromethoxy group, a difluoromethoxy group, a trifluoromethoxy group, or a 2,2,2-trifluoroethyl group but are a fluorine atom, a trifluoromethyl group, a fluoromethoxy group, a difluoromethoxy group, or a trifluoromethoxy group) as the liquid crystal compound having a positive dielectric anisotropy of 4 or more as an absolute value, and
the antioxidant according to claim 1.

7. The liquid crystal composition according to claim 6, comprising one or more kinds of polymerizable compounds.

8. A liquid crystal display device comprising:
a first substrate equipped with a common electrode formed of a transparent conductive material,
a second substrate equipped with a pixel electrode formed of a transparent conductive material and a thin film transistor for controlling the pixel electrode equipped on each pixel, and
a liquid crystal composition sandwiched between the first substrate and the second substrate, wherein
the alignment of liquid crystal molecules in the liquid crystal composition at the time of not applying a voltage is substantially parallel or substantially vertical to the substrates, and
the nematic liquid crystal composition according to claim 6 is used as the liquid crystal composition.

9. A liquid crystal display device which uses the liquid crystal composition according to claim 8 and is fabricated by polymerizing a polymerizable compound contained in the liquid crystal composition while applying a voltage or not applying a voltage.
